(19) United States
(12) Patent Application Publication
(10) Pub. No.: US 2008/0038735 A1
(43) Pub. Date: Feb. 14, 2008

(54) Tus DNA binding domains

(75) Inventors: Armin Sepp, Cambridge (GB); Albert Stoop, Cambridge (GB)

Correspondence Address: EDWARDS ANGELL PALMER & DODGE LLP, P.O. BOX 55874, BOSTON, MA, 02205, US

(73) Assignee: Domantis Limited, Cambridge (GB)

(21) Appl. No.: 11/728,574

(22) Filed: Mar. 26, 2007

Related U.S. Application Data
(63) Continuation-in-part of application No. PCT/GB05/04148, filed on Oct. 26, 2005.

(30) Foreign Application Priority Data
Oct. 27, 2004 (GB) 0423871.3

(51) Int. Cl. C12P 21/04; C07K 14/00; C12N 15/00; C12Q 1/68; C12N 15/11; C12N 5/06

(52) U.S. Cl. 435/6; 435/320.1; 435/325; 435/69.1; 530/350; 536/23.1

(57) ABSTRACT

The present invention relates to a nucleic sequence encoding one or more Tus DNA binding domains, one or more DNA binding sites and at least one polypeptide domain.

### RELATED APPLICATIONS

This is a continuation-in-part patent application which claims priority to PCT/GB2005/004148 filed on Oct. 26, 2005, which claims the benefit of GB 0423871.3 filed on Oct. 27, 2004. The entire teachings of the above applications are incorporated by reference.

### FIELD OF INVENTION

The present invention relates to the selection of polypeptide domains. In particular, the present invention relates to the selection of one or more polypeptide domains using a nucleotide sequence encoding one or more Tus DNA binding domains, one or more DNA binding sites and at least one polypeptide domain.

### BACKGROUND TO THE INVENTION

Evolution requires the generation of genetic diversity (diversity in nucleic acid) followed by the selection of those nucleic acids which result in beneficial characteristics. Because the nucleic acid and the activity of the encoded gene product of an organism are physically linked (the nucleic aids being confined within the sells which they encode) multiple rounds of mutation and selection can result in the progressive survival or organisms with increasing fitness. Systems for rapid evolution of nucleic acids or proteins in vitro should mimic this process at the molecular level in that the nucleic aid and the activity of the encoded gene product must be linked and the activity of the gene product must be selectable.

Recent advances in molecular biology have allowed some molecules to be co-selected according to their properties along with the nucleic acids that encode them. The selected nucleic acids can subsequently be closed for further analysis or use, or subjected to additional rounds of mutation and selection.

Common to these methods is the establishment of large libraries of nucleic acids. Molecules having the desired characteristics (activity) can be isolate through selection regimes that select for the desired activity of encoded gene product, such as a desired biochemical or biological activity, for example binding activity.

Phage display technology has been highly successful as providing a vehicle that allows for the selection of a displayed protein by providing the essential link between nucleic acid and the activity of the encoded gene product (Smith, 1985; Bass et al., 1990; McCafferty et al., 1990; for review see Clackson and Wells, 1994). Filamentous phage particles act as genetic display packages with proteins on the outside and the genetic elements, which encode them on the inside. The tight linkage between nucleic acid and the activity of the encoded gene product is a result of the assembly of the phage within bacteria. As individual bacteria are rarely multiply infected, in most cases all the phage produced from an individual bacterium will carry the same nucleotide sequence and display the same protein.

However, phage display relies upon the creation of nucleic acid libraries in vivo in bacteria. Thus, the practical limitation on library size allowed by phage display technology is of the order of 10⁷ to 10¹¹, even taking advantage of λ phage vectors with excisable filamentous phage replicons. The technique has mainly been applied to selection of molecules with binding activity. A small number of proteins with catalytic activity have also been isolated using this technique, however, in no case was selection directly for the desired catalytic activity, but either for binding to a transition-state analogue (Widersten and Mannervik, 1995) or reaction with a suicide inhibitor (Soumillion et al., 1994; Janda et al., 1997).

Another method is called Plasmid Display in which fusion proteins are expressed and folded within the E. coli cytoplasm and the phenotype-genotype linkage is created by the fusion proteins binding in vivo to DNA sequences on the encoding plasmids whilst still compartmentalised from other members of the library. In vitro selection from a protein library can then be performed and the plasmid DNA encoding the proteins can be recovered for re-transformation prior to characterisation or further selection. Specific peptide ligands have been selected for binding to receptors by affinity selection using large libraries of peptides linked to the C terminus of the lac repressor Lacl (Cull et al., 1992). When expressed in E. coli the repressor protein physically links the ligand to the encoding plasmid by binding to a lac operator sequence on the plasmid. Speight et al. (2001) describe a Plasmid Display method in which a nuclear factor κB p50 homodimer is used as a DNA binding protein which binds to a target κB site in the −10 region of a lac promoter. The protein-DNA complexes that are formed have improved stability and specificity.

An entirely in vitro polysome display system has also been reported (Mattheakis et al., 1994) in which nascent peptides are physically attached via the ribosome to the RNA which encodes them.

In vitro RNA selection and evolution (Ellington and Szostak, 1990), sometimes referred to as SELEX (systematic evolution of ligands by exponential enrichment) (Tuerk and Gold, 1990) allows for selection for both binding and chemical activity, but only for nucleic acids. When selection is for binding, a pool of nucleic acids is incubated with immobilised substrate. Non-binders are washed away, then the binders are released, amplified and the whole process is repeated in iterative steps to enrich for better binding sequences. This method can also be adapted to allow isolation of catalytic RNA and DNA (Green and Szostak, 1992; for reviews see Chapman and Szostak, 1994; Joyce, 1994; Gold et al., 1995; Moore, 1995).

WO99/02671 describes an in vitro sorting method for isolating one or more genetic elements encoding a gene product having a desired activity, comprising compartmentalising genetic elements into microcapsules; expressing the genetic elements to produce their respective gene products within the microcapsules; and sorting the genetic elements which produce the gene product having the desired activity. The invention enables the in vitro evolution of nucleic acids by repeated mutagenesis and iterative applications of the method of the invention.

In contrast to other methods WO99/02671 describes a man-made “evolution” system which can evolve both nucleic acids and proteins to effect the full range of biochemical and biological activities (for example, binding, catalytic and regulatory activities) and that can combine several processes leading to a desired product or activity.

A prerequisite for in vitro selection from large libraries of proteins is the ability to identify those members of the library with the desired activity (e.g. specificity). However, direct analysis of the selected protein requires much larger amounts of materials than are typically recovered in such experiments. One way in which this problem can be addressed involves the creation of a physical association between the encoding gene and the protein throughout the selection process and so the protein can be amplified and characterised by the encoding DNA or RNA.

The present invention seeks to provide an improved method for the in vitro selection of polypeptide domains according to their binding activity.

### SUMMARY OF THE INVENTION

The present invention relates, in part, to the surprising finding that Tus can be used for the in vitro selection of a polypeptide domain.

Thus, in a first aspect, the present invention relates to a nucleotide sequence encoding one or more Tus DNA binding domains, one or more DNA binding sites and at least one polypeptide domain.

The nucleotide sequence is expressed to produce its respective polypeptide domain gene product in fusion with the Tus DNA-binding domain. Once expressed, the polypeptide domain gene product becomes associated with its respective nucleotide sequence through the binding of the Tus DNA binding domain in the gene product to the DNA binding site-such as a Ter operator—of the respective nucleotide sequences. Typically, the nucleotide sequence of the present invention will be expressed within a microcapsule. The microcapsules comprising the nucleotide sequence can then be pooled into a common compartment in such a way that the nucleotide sequence bound to the polypeptide domain, preferably, an polypeptide domain (e.g. an antibody domain) with desirable properties (e.g. specificity or affinity), may be selected.

The nucleotide sequences according to the present invention may be cloned into a construct or a vector to allow further characterisation of the nucleotide sequences and their polypeptide domain gene products.

Thus, in a second aspect, the present invention relates to a construct comprising the nucleotide sequence according to the first aspect of the present invention.

In a third aspect; the present invention relates to a vector comprising the nucleotide sequence according to the first aspect of the present invention.

In a fourth aspect, the present invention relates to a host cell comprising the construct according to the second aspect of the present invention or the vector according to the third aspect of the present invention.

In a fifth aspect, the present invention relates to a protein encoded by the nucleotide sequence according to the first aspect of the present invention.

In a sixth aspect, the present invention relates to a protein-DNA complex comprising the protein according to the fifth aspect of the present invention bound to a nucleotide sequence according to the first aspect of the present invention—such as via one or more DNA binding sites.

Successful selection of polypeptide (e.g. antibody) domain-Tus fusion proteins on the basis of the antigen-binding activity depends among other factors also on the stability of the protein-DNA complex. The dissociation rate of the fusion protein-DNA interaction should be sufficiently low to maintain the genotype-phenotype linkage throughout the emulsion breakage and the subsequent affinity capture stage.

In a seventh aspect, the present invention relates to a method for preparing a protein-DNA complex according to the sixth aspect of the present invention, comprising the steps of: (a) providing a nucleotide sequence according to the first aspect of the present invention, a construct according to the second aspect of the present invention or a vector according to the third aspect of the present invention; and (b) expressing the nucleotide sequence to produce its respective protein, and (c) allowing for the formation of the protein-DNA complex.

In an eighth aspect, the present invention relates to a method for isolating one or more nucleotide sequences encoding a polypeptide domain with a desired specificity, comprising the steps of: (a) providing a nucleotide sequence according to the first aspect of the present invention, a construct according to the second aspect of the present invention or a vector according to the third aspect of the present invention; (b) compartmentalising the nucleotide sequence into microcapsules; (c) expressing the nucleotide sequence to produce its respective polypeptide domain; (d) pooling the microcapsules into a common compartment; and (e) selecting the nucleotide sequence which produces a polypeptide domain having the desired specificity.

The polypeptide domain nucleotide sequences are expressed to produce their respective polypeptide domain gene products within a microcapsule, such that the gene products are associated with the nucleotide sequences encoding them and the complexes thereby formed can be sorted. Advantageously, this allows for the nucleotide sequences and their associated gene products to be sorted according to the polypeptide domain specificity.

The nucleotide sequences may be sorted by a multi-step procedure, which involves at least two steps, for example, in order to allow the exposure of the polypeptide domain nucleotide sequences to conditions, which permit at least two separate reactions to occur. As will be apparent to a person skilled in the art, the first microencapsulation step must result in conditions which permit the expression of the polypeptide domain nucleotide sequences—be it transcription, transcription and/or translation, replication or the like. Under these conditions, it may not be possible to select for a particular polypeptide domain specificity, for example because the polypeptide domain may not be active under these conditions, or because the expression system contains an interfering activity.

Therefore, the selected polypeptide domain nucleotide sequence(s) may be subjected to subsequent, possibly more stringent rounds of sorting in iteratively repeated steps, reapplying the method of the present invention either in its entirety or in selected steps only. By tailoring the conditions appropriately, nucleotide sequences encoding polypeptide domain gene products having a better optimised specificity may be isolated after each round of selection.

The nucleotide sequence and the polypeptide domain thereby encoded are associated by confining each nucleotide sequence and the respective gene product encoded by the nucleotide sequence within the same microcapsule. In this way, the gene product in one microcapsule cannot cause a change in any other microcapsules.

Additionally, the polypeptide domain nucleotide sequences isolated after a first round of sorting may be subjected to mutagenesis before repeating the sorting by iterative repetition of the steps of the method of the invention as set out above. After each round of mutagenesis, some polypeptide domain nucleotide sequences will have been modified in such a way that the specificity of the gene products is enhanced.

In a ninth aspect, the present invention relates to a method for preparing a polypeptide domain, comprising the steps of: (a) providing a nucleotide sequence according to the first aspect of the present invention, a construct according to the second aspect of the present invention or a vector according to the third aspect of the present invention; (b) compartmentalising the nucleotide sequences; (c) expressing the nucleotide sequences to produce their respective gene products; (d) sorting the nucleotide sequences which produce polypeptide domains having the desired specificity; and (e) expressing the polypeptide domains having the desired specificity.

In a tenth aspect, the present invention relates to a protein-DNA complex obtained or obtainable by the method according to the seventh aspect of the present invention.

In an eleventh aspect, the present invention relates to a polypeptide domain obtained or obtainable by the method according to the eighth or ninth aspects of the present invention.

In an twelfth aspect, the present invention relates to the use of one or more Tus DNA binding domains and/or one or more Ter DNA binding sites in the selection of a polypeptide domain.

Preferably, the polypeptide domain is an antibody domain.

Preferably, the antibody domain is a V_{L}, V_{H} or Camelid V_{HH} domain.

Preferably, the nucleotide sequence comprises a tag sequence.

Preferably, the tag sequence is included at the 3′ end of the nucleotide sequence.

Preferably, the tag sequence is selected from the group consisting of HA, FLAG or c-Myc.

Preferably, the polypeptide domain is fused directly or indirectly to the N-terminus of the Tus DNA binding domain(s).

Preferably, the Tus DNA binding domain(s) comprises or consists of the sequence set forth in Seq ID No 1 or Seq ID No 2.

Preferably, the nucleotide sequence additionally comprises one or more linkers.

Preferably, the nucleotide sequence comprises 1, 2 or 3 DNA-binding sites.

Preferably, the one or more DNA-binding sites are Ter operator(s).

Preferably, the Ter operator(s) comprise or consist of TerB.

Preferably the Ter operator(s) comprise or consist of the sequence set forth in Seq ID No.3 or SEQ ID No. 4.

Preferably, the antibody domain is V_{κ}.

Preferably, the method according to the eighth aspect further comprises the additional step of: (f) introducing one or more mutations into the polypeptide domain.

Preferably, the method according to the eighth aspect further comprises iteratively repeating one or more of steps (a) to (e).

Preferably, the method according to the eighth aspect further comprises amplifying the polypeptide domain.

Preferably, the polypeptide domains are sorted by affinity purification.

Preferably, the polypeptide domains are sorted using protein L.

Preferably, the polypeptide domains are sorted by selective ablation of polypeptide domains, which do not encode the desired polypeptide domain gene product.

### DESCRIPTION OF THE FIGURES

FIG. 1

Schematic representation of the expression cassette of the pIE in vitro expression vectors where T7P denotes T7 promoter, g10e—g10 enhancer, RBS—ribosome binding site, ATG—Translation start site, HA—HA tag, TAA—STOP codon, T7T—T7 terminator. Also shown is the DNA sequence of the fragment of interest containing the cloning sites.

FIG. 2

Schematic representation showing insertion of the TUS gene in the BamHI site of the pIE2 vector. The TerB operator sequence has been inserted in the BglII site.

FIG. 3

The KEA linker was inserted in the NotI site of pIE2tT, thereby creating pIE7tT.

FIG. 4

Additional TerB operator sequences can be inserted in the BglII site, thereby creating the pIE7t³T series of vectors. By subsequently cloning Vₖ(E5) (SEQ ID No. 7) into the SalI-NotI site the final construct pIE7t³T.Vₖ(E5) was made.

FIG. 5

Binding of in vitro translated dAb-Tus fusion proteins to TNFa. A concentration range of TNFa is plotted against the ELISA signal obtained when the captured, in vitro translated, dAb-Tus fusion proteins were incubated with the indicated concentrations of biotinylated TNFa. TAR1-5-19 is the free dAb, 2tT(1-5-19) and 7tT(1-5-19) are TAR1-5-19 Vₖ domain antibodies fused to the Tus protein through either a A₃GS linker or a KEA linker, respectively.

FIG. 6

Binding of in vitro translated dAb-Tus fusion proteins to TerB operators. A concentration range of DNA is plotted against the ELISA signal obtained when captured, in vitro translated TAR(1-5-19)—Tus fusion proteins were incubated with the indicated concentrations of biotinylated TerB operator DNA. The 2tT vector contains the A₃GS linker while the 7tT vector contains the KEA linker. The captured, fusion proteins were incubated with either single (It) or triple (3t) TerB operator DNA.

FIG. 7

Time-dependent dissociation of TerB operator from TAR(1-5-19)-Tus fusion protein. In vitro translated TAR(1-5-19)—Tus fusion protein is incubated with biotinylated TerB operator DNA. After removal of the biotinylated DNA, dissociation of biotinylated operator is measured in time by determining the ELISA signal for the DNA at different time points. It and 3t denote single and triple TerB operator fragments. 2tT (A₃GS) and 7tT (KEA) denote the linker used to fuse TAR1-5-19 to Tus.

FIG. 8

Domain antibody and Tus function independently. ELISAs are performed in which in vitro translated pIE7tT(TAR1-5-19) is captured and incubated with biotinylated TNFa in presence and absence of excess amounts of DNA. Similarly, the fusion protein is incubated with biotinylated DNA (TerB operator) in the presence and absence of excess TNFa.

FIG. 9

Model selections without emulsification. Example in which a 1:100 mixture of TAR1-5-19:TAR1-5 in the pIE7t³T vector is subjected to selection with biotinylated TNFa. After capture on a streptavidin coated PCR plate, the bound DNA is amplified resulting in a product with a size specific for TAR1-5-19. If a 1:1 mixture is directly amplified, without selection, the smaller fragment, specific for TAR1-5, is predominantly amplified.

FIG. 10

Schematic representation of a model selection with emulsification. The DNA of pIE7t³T.Vₖ(X) and pIE7t³T.Vₖ(E5) are mixed in three different ratio's. After emulsification, selection and PCR with OA16 (SEQ ID No. 25) and OA17n (SEQ ID No. 26) single products are obtained (A). These are digested SalI-NotI, ligated in pIE7t³T and PCR amplified with AS16 (SEQ ID No. 18) and AS22 (B). These PCR products are in vitro translated and tested in an ELISA using a fixed amount of biotinylated cytokine A. The ELISA results after selection are plotted alongside a titration curve in C.

FIG. 11

Schematic representation of a single cycle of selection using emulsification and the Tus DNA binding protein.

FIG. 12

Schematic representation of the pUC119 GAS—myc vector used for expression of domain antibodies.

FIG. 13.

BIAcore analysis of Vₖ(X) and Vₖ(X*) for binding to cytokine A. On a streptavidin coated BIAcore chip, biotinylated cytokine A was captured. Subsequently, purified Vₖ(X) and Vₖ(X*) were injected and the association and dissociation of the dAbs to the cytokine were determined. The bottom line represents Vₖ(X) and the top curve represents Vₖ(X*).

FIG. 14

BIAcore analysis of Vk(Y) and Vk(Y*) for binding to Cytokine X. On a streptavidin coated BIAcore chip biotinylated Cytokine X was captured. Subsequently, purified Vk(Y) and Vk(Y*) were injected and the association and dissociation of the dAbs to Cytokine X were determined. The lower curve represents Vk(Y) and the top curve the improved variant Vk(Y*).

FIG. 15

BIAcore analysis of Vk(Z) and Vk(Z*) for binding to Cytokine Y. On a streptavidin coated BIAcore chip biotinylated Cytokine Y was captured. Subsequently, purified Vk(Z) and Vk(Z*) were injected and the association and dissociation of the dAbs to Cytokine Y were determined. The lower curve represents Vk(Z) and the top curve the improved variant Vk(Z*). The values indicate the dissociation constants (Kd) in nM for both domain antibodies as determined by BIAevaluation.

### DETAILED DESCRIPTION OF THE INVENTION

Polypeptide Domain

As used herein, the term “polypeptide domain” refers to a molecule or molecular construct that encodes a polypeptide domain—such as a V_{H} or a V_{L} domain.

In a preferred embodiment, the polypeptide domain is an antibody domain.

A typical antibody is a multi-subunit protein comprising four polypeptide chains; two “heavy” chains and two “light” chains. The heavy chain has four domains, the light chain has two domains. All of the domains are classified as either variable or constant.

The antigen binding domain of an antibody comprises two separate regions: a heavy chain variable domain (V_{H}) and a light chain variable domain (V_{L}: which can be either V_{κ} or V_{λ}).

The antigen-binding site itself is formed by six polypeptide loops: three from the V_{H} domain (H1, H2 and H3) and three from the V_{L} domain (L1, L2 and L3).

The V_{H} gene is produced by the recombination of three gene segments, V_{H}, D and J_{H}. In humans, there are approximately 51 functional V_{H} segments (Cook and Tomlinson (1995) Immunol Today, 16: 237), 25 functional D segments (Corbett et al. (1997) J. Mol. Biol., 268: 69) and 6 functional J_{H} segments (Ravetch et al. (1981) Cell, 27: 583), depending on the haplotype. The V_{H} segment encodes the region of the polypeptide chain which forms the first and second antigen binding loops of the V_{H} domain (H1 and H2), whilst the V_{H}; D and J_{H} segments combine to form the third antigen binding loop of the V_{H} domain (H3).

The V_{L} gene is produced by the recombination of two gene segments, V_{L} and J_{L}. In humans, there are approximately 40 functional V_{κ} segments (Schäble and Zachau (1993) Biol. Chem. Hoppe-Seyler, 374: 1001), 31 functional V_{λ} segments (Williams et al. (1996) J. Mol. Biol., 264: 220; Kawasaki et al. (1997) Genome Res., 7: 250), 5 functional J_{κ} segments (Hieter et al. (1982) J. Biol. Chem., 257: 1516) and 4 functional J_{λ} segments (Vasicek and Leder (1990) J. Exp. Med., 172: 609), depending on the haplotype. The V_{L} segment encodes the region of the polypeptide chain which forms the first and second antigen binding loops of the V_{L} domain (L1 and L2), whilst the V_{L} and J_{L} segments combine to form the third antigen binding loop of the V_{L} domain (L3). Antibodies selected from this primary repertoire are believed to be sufficiently diverse to bind almost all antigens with at least moderate affinity. High affinity antibodies are produced by “affinity maturation” of the rearranged genes, in which point mutations are generated and selected by the immune system on the basis of improved binding.

The polypeptide domains may be provided in the form of a library.

Typically, the antibody domains will be provided in the form of a library, which will in most cases require the screening of a large number of variant antibody domains. Libraries of antibody domains may be created in a variety of different ways, including the following.

Pools of naturally occurring antibody domains may be cloned from genomic DNA or cDNA (Sambrook et al., 1989); for example, phage antibody libraries, made by PCR amplification repertoires of antibody genes from immunised or unimmunised donors have proved very effective sources of functional antibody fragments (Winter et al., 1994; Hoogenboom, 1997). Libraries of genes encoding antibody domains may also be made by encoding all (see for example Smith, 1985; Parmley and Smith, 1988) or part of genes (see for example Lowman et al., 1991) or pools of genes (see for example Nissim et al., 1994) by a randomised or doped synthetic oligonucleotide. Libraries may also be made by introducing mutations into an antibody domain or pool of antibody domains ‘randomly’ by a variety of techniques in vivo, including; using ‘mutator strains’, of bacteria such as E. coli mutD5 (Liao et al., 1986; Yamagishi et al., 1990; Low et al., 1996); and using the antibody hypermutation system of B-lymphocytes (Yelamos et al., 1995). Random mutations can also be introduced both in vivo and in vitro by chemical mutagens, and ionising or UV irradiation (see Friedberg et al., 1995), or incorporation of mutagenic base analogues (Freese, 1959; Zaccolo et al., 1996). ‘Random’ mutations can also be introduced into antibody domains genes in vitro during polymerisation for example by using error-prone polymerases (Leung et al., 1989).

Further diversification may be introduced by using homologous recombination either in vivo (see Kowalczykowski et al., 1994 or in vitro (Stemmer, 1994a; Stemmer, 1994b)).

Preferably, the antibody domain is a V_{H} or a V_{L} antibody domain.

The antibody domain may be a Camelid VHH domain (i.e. a V domain derived or derivable from a Camelid antibody consisting of two heavy chains).

The antibody domain may be part of a monoclonal antibody (mAb), e.g. V_{L} or V_{κ} single-domain antibody (dAb). dAbs are described in Ward et al. (1989) Nature 341, p 544-546. Preferably, the antibody V_{L} domain is V_{κ}.

The polypeptide domain may be fused directly or indirectly to the N-terminus of the Tus DNA binding domain(s).

In this context, the term “directly” means that the polypeptide domain is fused to the Tus DNA binding domain(s) in the absence of a linker.

In this context, the term “indirectly” means that the polypeptide domain is fused to the Tus DNA binding domain(s) via at least a linker.

Preferably, the polypeptide domain is fused indirectly to the N-terminus of the Tus DNA binding domain(s).

Typically, the DNA binding site will be located at the 5′ end of the nucleotide sequence.

Variable domains may even be linked together to form multivalent ligands by, for example: provision of a hinge region at the C-terminus of each V domain and disulphide bonding between cysteines in the hinge regions.

DNA-Binding Domains

The DNA-binding domain that provides the genotype-phenotype linkage in an emulsion-based in vitro selection should satisfy several criteria.

The DNA-binding proteins should form a highly stable protein-DNA complex in the in vitro translation mix. High stability means in this context, a very low dissociation rate constant such that the genotype-phenotype linkage between a gene and its encoded protein product is faithfully maintained throughout the processes of breaking the emulsion and the affinity capture of the protein-DNA complexes with desired properties. Typically, the genotype-linkage should be maintained at an acceptable level for at least approximately ten minutes, meaning that the dissociation rate constant should be at least in the region of 10⁻³ s⁻¹ or smaller.

It can be advantageous if the DNA-binding domain does not substantially interfere with the binding properties of the polypeptide domain. It can be advantageous if the DNA-binding domain loses (if at all) only a limited amount of DNA-binding activity in the fusion protein format. It can also be advantageous if the DNA-binding protein does not have any Cystein residues (either reduced or oxidised) in the functionally active form of the fusion protein. Cystein residues in the DNA-binding domain of the fusion protein format may interfere with the intradomain oxidation of the cystein residues of the polypeptide (e.g. antibody) domain. Additionally, the redox conditions which are optimal for in vitro expression may not be optimal for the DNA binding domain.

Many different DNA-binding proteins have been identified from species ranging from bacteria to vertebrates. As of July 2001, the SWISS-PROT database (Release 38) contained 3238 full-length sequences which contained at least one DNA-binding domain. These 3238 sequences were further classified into 22 structurally related families (Karmirantzou & Hamodrakas (2001). Many of these DNA-binding proteins have been studied in great detail, including binding characteristics and three-dimensional structures, often in complex with DNA fragments bearing cognate binding sites (Karmirantzou & Hamodrakas (2001). For example, among the best-studied DNA-binding proteins with lower Kd values are Zn-finger proteins, e.g. TFIIIA from Xenopus (Miller et al., 1985) and Arc repressor from phage P22 (Raumann et al. (1994)).

The consensus sequence for the TFIIIA-type zinc finger domains is Tyr/Phe-X-Cys-X24-Cys-X3-Phe-X5-Leu-X2-His-X3-5-His (where X is any amino acid). As a rule there are from 2 up to 37 Zn-finger domains per protein, usually arranged in tandem. Each zinc finger is an autonomously folding mini-domain, which is dependent on a zinc ion for stability. The tertiary structure of a typical Zn-finger domain is comprised of an anti parallel β-sheet packed against a predominantly α-helical domain, with the invariant cysteines and histidines chelating the zinc ion and the three conserved hydrophobic residues forming a core (Choo & Klug (1993)). However, although extremely high-affinity Zn-finger proteins have been designed and characterised, with Kd values in low pM range, these proteins require the presence of 5 mM DTT for the preservation of functional activity (Moore et al. (2001)). Such strongly reducing conditions are unsuitable for the in vitro expression of antibody fragments, as demonstrated in the case of single-chain antibodies (Ryabova & Desplancq, et al. (1997)).

The wild-type Arc repressor from the P22 bacteriophage is a member of the ribbon-helix-helix family of transcription factors which controls transcription during the lytic growth of bacteriophage P22 by binding to the semi-palindromic Arc operator as a dimer of dimers. Each Arc dimer uses an antiparallel beta-sheet to recognize bases in the major groove whilst a different part of the protein surface is involved in dimer-dimer interactions. At high concentrations, the Arc repressor is a reasonably stable dimer. However, at the sub-nanomolar concentrations where half-maximal operator binding is observed, Arc dimers disassociate and most molecules exist as unfolded monomers.

In general, there may be more than one DNA binding site present on the genetic elements allowing the binding of multiple copies of the fusion protein. Such multiplication of the identical copies of protein molecules encoded by a given gene can be used to harness the avidity effect in antibody-antigen interactions, since the number of polypeptide domains associated with a DNA protein increases too when the number of DNA-bound protein molecules increases.

Surprisingly, it has been found that the Tus DNA binding domain can be used for the selection of one or more polypeptide domains.

Advantageously, a small non-interacting DNA stuffer fragment may be inserted between the Tus DNA binding domain(s) and the T7 promoter. This makes it possible to identify rapidly the polypeptide domain—such as dAb—by the size of the PCR product that is obtained.

Tus DNA Binding Domain

As used herein, the term “Tus DNA-binding domain” refers to a domain of a Tus DNA binding protein that is required for the protein to bind to a DNA binding site—such as a Ter operator. The binding between the Tus DNA binding protein(s) and the DNA binding site(s) will be maintained throughout the emulsion breakage and the subsequent affinity capture stage, preferably for about at least 1 hour.

The Tus protein (E. coli DNA replication terminus site binding protein) terminates replication of DNA in E. coli and consists of two α-helical bundles at the amino and carboxy termini, connected by a large β-sheet region and binds DNA as a monomer. The DNA-binding region of the Tus family is made of four antiparallel β strands which links the amino- and carboxy-terminal domains and produces a large central cleft in the protein. The DNA is bound in this cleft, with the inter-domain β strands contacting bases in the major groove. DNA backbone contacts are provided by the whole protein. The β strands are positioned almost perpendicular to the base edges in the groove, enabling contacts from amino acids that expose their side chains on either face of the sheet (Kamada et al. (1996) Nature 383, p 598-603).

The tus gene is located immediately adjacent to the TerB site. The Tus DNA-binding protein comprises 309 amino acids (35.8 kilodaltons) that have no apparent homology to the helix-turn-helix, zinc finger, or leucine zipper motifs of other DNA-binding proteins. Binding of Tus arrests DNA replication at the second base pair of the Ter site by preventing DNA unwinding by the DnaJ3 helicase. The equilibrium binding constant (K_{D}) for the Tus DNA binding protein is 0.34 pM. The half life of a Tus-DNA complex is about 550 min., with a dissociation rate constant of 2.1−7.7×10⁻⁵ s⁻¹ and an association rate constant of 1.0−1.4×10⁻⁸ M⁻¹ s⁻¹ (Gottlieb et al. (1992) J. Biol. Chem. 267, p 7434-7443 and Skokotas et al., (1995) J Biol Chem. 29; 270(52):30941-8).

Preferably, the Tus DNA binding domain(s) comprises or consists of the sequence set forth in Seq ID No 1 or Seq ID No 2 (as set forth in J. Biol. Chem. (1989) 264 (35), 21031-21037) or a variant, homologue, fragment or derivative thereof.

The sequence of the Tus DNA binding domain(s) may be modified (e.g. mutated) to modulate the degree of binding.

Accordingly, mutated Tus DNA binding domain(s) are also contemplated provided that such mutants have Tus DNA binding domain activity, preferably being at least as biologically active as the Tus DNA binding domain from which the mutated sequence was derived. Preferably, if the sequence of the Tus DNA binding domain(s) is modified, then the degree of binding is increased.

The nucleotide sequence according to the present invention may comprise one or more Tus DNA-binding domains, for example, 1, 2 or 3 or more Tus DNA-binding domains. Preferably, the nucleotide sequence according to the present invention comprises one Tus DNA-binding domains.

A plurality of Tus DNA binding domains may be obtained by designing a recombinant gene containing tandem copies of the Tus DNA binding domain(s) coding sequence with intervening DNA encoding a sequence to join the Tus DNA binding domain(s). Preferably, this sequence joins the C-terminus of one Tus DNA binding domain monomer to the N-terminus of the next Tus DNA binding domain.

The Tus DNA binding domain(s) may be joined by a linker.

The Tus DNA binding domain(s) may be adjacent to a promoter—such as a T7 promoter.

Methods for obtaining novel DNA-binding proteins have been described in the art. By way of example, novel DNA-binding proteins that preferentially bind a predetermined DNA sequence in double stranded DNA are described in U.S. Pat. No. 5,096,815. Mutated genes that specify novel proteins with desirable sequence-specific DNA-binding properties are separated from closely related genes that specify proteins with no or undesirable DNA-binding properties.

A person skilled in the art will appreciate that such methods may be used to design novel Tus DNA-binding proteins—such as novel Tus repressors. Advantageously, novel Tus DNA-binding proteins that bind specific DNA sequence motifs—such as wild type or mutated DNA binding sites—may be used in the present invention.

The activity of a Tus DNA binding domain(s) may be determined using various methods in the art—such as those described in Gottlieb et al. (1992) J. Biol. Chem. 267, p 7434-7443. Briefly the assay for binding to single-stranded DNA is assessed using a polyacrylamide gel shift assay. Individual strands are labelled with T4 DNA kinase and [y-32P]ATP for 10 min at 37° C. The excess ATP is removed by size exclusion column chromatography. Twenty fmol of labelled DNA are then mixed with Tus protein in a final volume of 20 μl in KG binding buffer. Samples are incubated for 30 min at 25° C., and to this solution is added 5 μl of a dye solution containing 0.125 M EDTA, 50% glycerol, 0.1% xylene cyanol, and 0.1% bromphenol blue. The samples are immediately loaded onto a 5% polyacrylamide gel containing TE buffer (20 mM Tris-C1, pH 7.5, 1 mM EDTA) and electrophoresed at 15 V/cm for 1.5 h with continuous buffer circulation. The gels were then dried and exposed to film.

DNA Binding Site

The term “DNA binding site” refers to a DNA sequence to which a Tus DNA-binding domain can bind.

Preferably, the DNA-binding domain can bind with high affinity and specificity.

Preferably, the term “DNA binding site” refers to a Ter operator to which a Tus DNA-binding domain binds.

Various Ter operators have been described in the art, for example, TerA, TerB (Hill et al., (1987) PNAS 84, p 1754-1758; deMassy et al., (1987) PNAS 84, 1759-1763), TerC, TerD (Hidaka et al., (1988) Cell 55 p 467-475; Francois et al. (1989) Mol. Mirobiol. 3, 995-1002), TerE (Hidaka et al., (1991) J. Bacteriol. 173 p 391-393) and TerF have been identified. The Ter sites consist of 23 base pair sequences that lack the dyad symmetry commonly found in other DNA-binding sites. Ter sites have also been identified in other replicons—such as the plasmids R6K and R100 (Kolter and Helinski (1978) J. Mol. Biol. 124 p 425-441; Bastia et al., (1981) Gene 14 p 81-89; Horiuchi and Hidaka (1988) Cell 54, p 515-523; Hill et al. (1988b) Cell 55 459-466), Salmonella typhimurium (Roecklein et al., (1991) Res. Microbiol. 142, p 169-176), and Bacillus shtilis (Weiss and Wake (1984) J. Mol. Biol. 179, 745-750; Lewis et al. (1990) J. Mol. Biol. 214, p 72-84).

Preferably, the DNA binding site is a TerB operator

Preferably, the DNA binding site(s) comprises the sequence shown in Seq ID No. 3 or SEQ ID No. 4 or a variant, homologue fragment or derivative thereof.

Preferably, the DNA binding site(s) consists of the sequence shown in Seq ID No. 3 or SEQ ID No. 4 or a variant, homologue fragment or derivative thereof.

In general, nucleotide sequences containing the following variation will also work:

(a/n)gn(a/g)(t/n)gttgtaa(c/t)(t/g)a(a/n), wherein n=a, t, c or g

as described by Coskun-Ari & Hill TM (J Biol. Chem. (1997) 17 272(42):26448-56).

The nucleotide sequence may comprise 1, 2 or 3 or more DNA binding sites.

In a preferred embodiment, the nucleotide sequence comprises 1, 2 or 3 DNA binding sites.

When 3 operators are used, the protein-DNA complex is stable for greater than 5 hours.

In a further preferred embodiment, the nucleotide sequence comprises 1 DNA binding sites. Therefore, in this embodiment, the binding of the Tus DNA binding domain is monomeric and binds to a single DNA binding site. This ensures binding of a single Tus DNA binding domain and the selection of a single polypeptide. One advantage of this format over, for example, scArc is the ability of the system to be monomeric, whereas the scArc system is at least dimeric and when multiple operators are used, tetrameric etc. Monomeric presentation is advantageous because, for example, many antigens are multimeric and so presentation of dAbs in a multimeric fashion—such as using scArc or phage—will lead to various avidity effects and thus obscure the isolation of high affinity binders.

Typically, the distance between the operator sites will be about 19 base pairs. This corresponds to approximately one and a half helical turns of the DNA helix.

The sequence of the DNA binding site(s) may be modified (e.g. mutated) to modulate the degree of binding to the Tus DNA binding domain(s). Preferably, if the sequence of the DNA binding site(s) is modified, then the degree of binding to the Tus DNA binding domain(s) is substantially the same or is increased as compared to the unmodified DNA binding site.

Tag Sequence

As used herein the term “tag sequence” refers to one or more additional sequences that are added to facilitate protein purification and/or isolation.

Examples of tag sequences include glutathione-S-transferase (GST), 6×His, GAL4 (DNA binding and/or transcriptional activation domains), β-galactosidase, the C-myc motif, the anti-FLAG-tag or the HA tag. It may also be convenient to include a proteolytic cleavage site between the tag sequence and the protein sequence of interest to allow removal of fusion protein sequences.

Preferably, the fusion protein will not hinder the activity of the protein sequence.

Advantageously, epitope tags are used which can be easily detected and purified by immunological methods. A unique tag sequence is added to the nucleotide sequence by recombinant DNA techniques, creating a fusion protein that can be recognised by an antibody specific for the tag peptide. The major advantage of epitope tagging is the small size of the added peptide sequences, usually 3 to 12 amino acids, which generally have no effect on the biological function of the tagged protein. In addition, for most biochemical applications, the use of epitope tags eliminates the need to generate an antibody to the specific protein being studied.

A preferred tag sequence is the HA tag, which is a nine amino acid peptide sequence (YPYDVPDYA) present in the human influenza virus hemagglutinin protein.

The HA tag is recognised by an anti-HA antibody as described herein. The HA tag has been successfully fused to proteins at their amino terminal end, carboxy terminal end, or at various sites within the target protein sequence. In addition, HA-tagged proteins may be expressed and detected in bacteria, yeast, insect cells, and mammalian cells.

Preferably, the tag sequence is located at the 3′ end of the nucleotide sequence.

Optionally, a linker may be located between the 3′ end of the nucleotide sequence and the tag sequence.

Linker

Preferably, a linker separates the polypeptide domain(s) and the Tus DNA binding domain(s).

If more than one Tus DNA binding domain is included in the construct, then a linker may even separate the Tus DNA binding domains.

The sequence of the linker may be based upon those used in the construction of single-chain antigen binding proteins (Methods Enzymol. (1991) 203, 36-89). Typically, the sequence will be chosen to maximises flexibility and solubility and allow the introduction of restriction sites for cloning and gene construction. Such sequences may be designed using the methods described in Biochemistry (1996) 35, 109-116 and may even comprise the sequences set forth therein.

The linker may comprise any amino acid.

The linker may comprise or consist of the sequence (GₙS)ₙ. The linker may comprise or consist of the sequence (Gₙ₁,S)ₙ₂, wherein n1 is from 1-3 and n2 is 1 or 2, preferably, n1 is 3 and n2 is 2. The linker may comprise or consist of the sequence (Gₙ₁S)ₙ₂, wherein n1 is from 1-3 and n2 is from 1-7, preferably, n1 is 3 and n2 is 7.

The linker may comprise the sequence (KEAₙ₁)ₙ₂, wherein n1=1-3 and n2=1-8, preferably, n1=3 and n2=8. Preferably, this linker comprises or consists of the sequence set forth in SEQ ID No. 8 or SEQ ID No. 9 (PNAS (1987) 84, 8898-8902; Protein Engineering (2001), 14, 529-532).

The linker may comprise or consist of the sequence (AₙGS), wherein n=1-3, preferably n=3.

A person skilled in the art will appreciate that other suitable linker sequences may be designed using the methods described in, for example, Biochemistry (1996) 35, 109-116.

Nucleotide Sequence

The nucleotide sequence according to the present invention may comprise any nucleic acid (for example, DNA, RNA or any analogue, natural or artificial, thereof).

The DNA or RNA may be of genomic or synthetic or of recombinant origin (e.g. cDNA), or combinations thereof.

The nucleotide sequence may be double-stranded or single-stranded whether representing the sense strand or the antisense strand or combinations thereof. The nucleotide sequence may be a gene.

Preferably, the nucleotide sequence is selected from the group consisting of a DNA molecule, an RNA molecule, a partially or wholly artificial nucleic acid molecule consisting of exclusively synthetic or a mixture of naturally-occurring and synthetic bases, any one of the foregoing linked to a polypeptide, and any one of the foregoing linked to any other molecular group or construct.

The one or more Tus DNA binding domains, one or more DNA binding sites and at least one polypeptide domain, and optionally, the tag and/or linker sequences, are operably linked.

As used herein, the term “operably linked” refers to a juxtaposition wherein the nucleotide sequences are joined (e.g. ligated) together in a relationship that permits them to be expressed as an expression product (e.g. a gene product).

The nucleotide sequence may comprise suitable regulatory sequences, such as those required for efficient expression of the gene product, for example promoters, enhancers, translational initiation sequences and the like.

The nucleotide sequence may moreover be linked, covalently or non-covalently, to one or more molecules or structures, including proteins, chemical entities and groups, solid-phase supports and the like.

Expression

Expression, as used herein, is used in its broadest meaning, to signify that a nucleotide sequence is converted into its gene product.

Thus, where the nucleic acid is DNA, expression refers to the transcription of the DNA into RNA; where this RNA codes for protein, expression may also refer to the translation of the RNA into protein. Where the nucleic acid is RNA, expression may refer to the replication of this RNA into further RNA copies, the reverse transcription of the RNA into DNA and optionally the transcription of this DNA into further RNA molecule(s), as well as optionally the translation of any of the RNA species produced into protein.

Preferably, therefore, expression is performed by one or more processes selected from the group consisting of transcription, reverse transcription, replication and translation.

Expression of the nucleotide sequence may thus be directed into either DNA, RNA or protein, or a nucleic acid or protein containing unnatural bases or amino acids (the gene product), preferably within the microcapsule of the invention, so that the gene product is confined within the same microcapsule as the nucleotide sequence.

Microcapsule

As used herein, the term “microcapsule” refers to a compartment whose delimiting borders restrict the exchange of the components of the molecular mechanisms described herein which allow the sorting of nucleotide sequences according to the specificity of the polypeptide (e.g. antibody) domains which they encode.

The microcapsule may be a cell—such as a yeast, fungal or bacterial cell. If the cell is a bacterial cell then it may be in the form of a spheroplast. Spheroplasts may be prepared using various methods in the art. By way of example, they may be prepared by resuspending pelleted cells in a buffer containing sucrose and lysozyme.

Preferably, the microcapsule is artificial.

Preferably, the microcapsules used in the methods of the present invention will be capable of being produced in very large numbers, and thereby able to compartmentalise a library of nucleotide sequences which encode a repertoire of polypeptide domains, for example, antibody domains

The microcapsules of the present invention require appropriate physical properties to allow them to work successfully.

First, to ensure that the nucleotide sequences and gene products do not diffuse between microcapsules, the contents of each microcapsule must be isolated from the contents of the surrounding microcapsules, so that there is no or little exchange of the nucleotide sequences and gene products between the microcapsules over the timescale of the experiment.

Second, there should be only a limited number of nucleotide sequences per microcapsule. This ensures that the gene product of an individual nucleotide sequence will be isolated from other nucleotide sequences. Thus, coupling between nucleotide sequence and gene product will be highly specific. The enrichment factor is greatest with on average one or fewer nucleotide sequences per microcapsule, the linkage between nucleic acid and the activity of the encoded gene product being as tight as is possible, since the gene product of an individual nucleotide sequence will be isolated from the products of all other nucleotide sequences. However, even if the theoretically optimal situation of, on average, a single nucleotide sequence or less per microcapsule is not used, a ratio of 5, 10, 50, 100 or 1000 or more nucleotide sequences per microcapsule may prove beneficial in sorting a large library. Subsequent rounds of sorting, including renewed encapsulation with differing nucleotide sequence distribution, will permit more stringent sorting of the nucleotide sequences. Preferably, there is a single nucleotide sequence, or fewer, per microcapsule.

Third, the formation and the composition of the microcapsules must not abolish the function of the machinery for the expression of the nucleotide sequences and the activity of the gene products.

Consequently, any microencapsulation system used should fulfil these three requirements. The appropriate system(s) may vary depending on the precise nature of the requirements in each application of the invention, as will be apparent to the skilled person.

A wide variety of microencapsulation procedures are available (see Benita, 1996) and may be used to create the microcapsules used in accordance with the present invention. Indeed, more than 200 microencapsulation methods have been identified in the literature (Finch, 1993).

These include membrane enveloped aqueous vesicles such as lipid vesicles (liposomes) (New, 1990) and non-ionic surfactant vesicles (van Hal et al., 1996). These are closed-membranous capsules of single or multiple bilayers of non-covalently assembled molecules, with each bilayer separated from its neighbour by an aqueous compartment. In the case of liposomes the membrane is composed of lipid molecules; these are usually phospholipids but sterols such as cholesterol may also be incorporated into the membranes (New, 1990). A variety of enzyme-catalysed biochemical reactions, including RNA and DNA polymerisation, can be performed within liposomes (Chakrabarti et al., 1994; Oberholzer et al., 1995a; Oberholzer et al., 1995b; Walde et al., 1994; Wick & Luisi, 1996).

With a membrane-enveloped vesicle system much of the aqueous phase is outside the vesicles and is therefore non-compartmentalised. This continuous, aqueous phase should be removed or the biological systems in it inhibited or destroyed (for example, by digestion of nucleic acids with DNase or RNase) in order that the reactions are limited to the microcapsules (Luisi et al., 1987).

Enzyme-catalysed biochemical reactions have also been demonstrated in microcapsules generated by a variety of other methods. Many enzymes are active in reverse micellar solutions (Bru & Walde, 1991; Bru & Walde, 1993; Creagh et al., 1993; Haber et al., 1993; Kumar et al., 1989; Luisi & B., 1987; Mao & Walde, 1991; Mao et al., 1992; Perez et al., 1992; Walde et al., 1994; Walde et al., 1993; Walde et al., 1988) such as the AOT-isooctane-water system (Menger & Yamada, 1979).

Microcapsules can also be generated by interfacial polymerisation and interfacial complexation (Whateley, 1996). Microcapsules of this sort can have rigid, nonpermeable membranes, or semipermeable membranes. Semipermeable microcapsules bordered by cellulose nitrate membranes, polyamide membranes and lipid-polyamide membranes can all support biochemical reactions, including multienzyme systems (Chang, 1987; Chang, 1992; Lim, 1984). Alginate/polylysine microcapsules (Lim & Sun, 1980), which can be formed under very mild conditions, have also proven to be very biocompatible, providing, for example, an effective method of encapsulating living cells and tissues (Chang, 1992; Sun et al., 1992).

Non-membranous microencapsulation systems based on phase partitioning of an aqueous environment in a colloidal system, such as an emulsion, may also be used.

Preferably, the microcapsules of the present invention are formed from emulsions; heterogeneous systems of two immiscible liquid phases with one of the phases dispersed in the other as droplets of microscopic or colloidal size (Becher, 1957; Sherman, 1968; Lissant, 1974; Lissant, 1984).

Emulsions may be produced from any suitable combination of immiscible liquids. Preferably the emulsion has water (containing the biochemical components) as the phase present in the form of finely divided droplets (the disperse, internal or discontinuous phase) and a hydrophobic, immiscible liquid (an ‘oil’) as the matrix in which these droplets are suspended (the nondisperse, continuous or external phase). Such emulsions are termed ‘water-in-oil’ (W/O). This has the advantage that the entire aqueous phase containing the biochemical components is compartmentalised in discreet droplets (the internal phase). The external phase, being a hydrophobic oil, generally contains none of the biochemical components and hence is inert.

The emulsion may be stabilised by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents (Ash and Ash, 1993). Suitable oils include light white mineral oil and non-ionic surfactants (Schick, 1966) such as sorbitan monooleate (Span™80; ICI) and t-octylphenoxypolyethoxyethanol (Triton X-100, Sigma).

The use of anionic surfactants may also be beneficial. Suitable surfactants include sodium cholate and sodium taurocholate. Particularly preferred is sodium deoxycholate, preferably at a concentration of 0.5% w/v, or below. Inclusion of such surfactants can in some cases increase the expression of the nucleotide sequences and/or the activity of the gene products. Addition of some anionic surfactants to a non-emulsified reaction mixture completely abolishes translation. During emulsification, however, the surfactant is transferred from the aqueous phase into the interface and activity is restored. Addition of an anionic surfactant to the mixtures to be emulsified ensures that reactions proceed only after compartmentalisation.

Creation of an emulsion generally requires the application of mechanical energy to force the phases together. There are a variety of ways of doing this which utilise a variety of mechanical devices, including stirrers (such as magnetic stir-bars, propeller and turbine stirrers, paddle devices and whisks), homogenisers (including rotor-stator homogenisers, high-pressure valve homogenisers and jet homogenisers), colloid mills, ultrasound and ‘membrane emulsification’ devices (Becher, 1957; Dickinson, 1994).

Aqueous microcapsules formed in water-in-oil emulsions are generally stable with little if any exchange of nucleotide sequences or gene products between microcapsules. Additionally, we have demonstrated that several biochemical reactions proceed in emulsion microcapsules. Moreover, complicated biochemical processes, notably gene transcription and translation are also active in emulsion microcapsules. The technology exists to create emulsions with volumes all the way up to industrial scales of thousands of litres (Becher, 1957; Sherman, 1968; Lissant, 1974; Lissant, 1984).

The preferred microcapsule size will vary depending upon the precise requirements of any individual selection process that is to be performed according to the present invention. In all cases, there will be an optimal balance between gene library size, the required enrichment and the required concentration of components in the individual microcapsules to achieve efficient expression and reactivity of the gene products.

The processes of expression must occur within each individual microcapsule provided by the present invention. Both in vitro transcription and coupled transcription-translation become less efficient at sub-nanomolar DNA concentrations. Because of the requirement for only a limited number of DNA molecules to be present in each microcapsule, this therefore sets a practical upper limit on the possible microcapsule size. Preferably, the mean volume of the microcapsules is less that 5.2×10⁻¹⁶ m³, (corresponding to a spherical microcapsule of diameter less than 10 μm, more preferably less than 6.5×10⁻¹⁷ m³ (5 μm), more preferably about 4.2×10⁻¹⁸ m³ (2 μm) and ideally about 9×10⁻¹⁸ m³ (2.6 μm).

The effective DNA or RNA concentration in the microcapsules may be artificially increased by various methods that will be well-known to those versed in the art. These include, for example, the addition of volume excluding chemicals such as polyethylene glycols (PEG) and a variety of gene amplification techniques, including transcription using RNA polymerases including those from bacteria such as E. coli (Roberts, 1969; Blattner and Dahlberg, 1972; Roberts et al., 1975; Rosenberg et al., 1975), eukaryotes e.g. (Weil et al., 1979; Manley et al., 1983) and bacteriophage such as T7, T3 and SP6 (Melton et al., 1984); the polymerase chain reaction (PCR) (Saiki et al., 1988); Qβ replicase amplification (Miele et al., 1983; Cahill et al., 1991; Chetverin and Spirin, 1995; Katanaev et al., 1995); the ligase chain reaction (LCR) (Landegren et al., 1988; Barany, 1991); and self-sustained sequence replication system (Fahy et al., 1991) and strand displacement amplification (Walker et al., 1992). Even gene amplification techniques requiring thermal cycling such as PCR and LCR could be used if the emulsions and the in vitro transcription or coupled transcription-translation systems are thermostable (for example, the coupled transcription-translation systems could be made from a thermostable organism such as Thermus aquaticus).

Increasing the effective local nucleic acid concentration enables larger microcapsules to be used effectively. This allows a preferred practical upper limit to the microcapsule volume of about 5.2×10⁻¹⁶ m³ (corresponding to a sphere of diameter 10 μm).

The microcapsule size must be sufficiently large to accommodate all of the required components of the biochemical reactions that are needed to occur within the microcapsule. For example, in vitro, both transcription reactions and coupled transcription-translation reactions require a total nucleoside triphosphate concentration of about 2 mM.

For example, in order to transcribe a gene to a single short RNA molecule of 500 bases in length, this would require a minimum of 500 molecules of nucleoside triphosphate per microcapsule (8.33×10⁻²² moles). In order to constitute a 2 mM solution, this number of molecules must be contained within a microcapsule of volume 4.17×10⁻¹⁹ litres (4.17×10⁻²² m³ which if spherical would have a diameter of 93 nm.

Furthermore, particularly in the case of reactions involving translation, it is to be noted that the ribosomes necessary for the translation to occur are themselves approximately 20 nm in diameter. Hence, the preferred lower limit for microcapsules is a diameter of approximately 0.1 μm (100 nm).

Therefore, the microcapsule volume is preferably of the order of between 5.2×10⁻²² m³ and 5.2×10⁻¹⁶ m³ corresponding to a sphere of diameter between 0.1 μm and 10 μm, more preferably of between about 5.2×10⁻¹⁹ m³ and 6.5×10⁻¹⁷ m³ (1 μm and 5 μm). Sphere diameters of about 2.6 μm are most advantageous.

It is no coincidence that the preferred dimensions of the compartments (droplets of 2.6 μm mean diameter) closely resemble those of bacteria, for example, Escherichia are 1.1−1.5×2.0−6.0 μm rods and Azotobacter are 1.5-2.0 μm diameter ovoid cells. In its simplest form, Darwinian evolution is based on a ‘one genotype one phenotype’ mechanism. The concentration of a single compartmentalised gene, or genome, drops from 0.4 nM in a compartment of 2 μm diameter, to 25 pM in a compartment of 5 μm diameter. The prokaryotic transcription/translation machinery has evolved to operate in compartments of ˜1-2 μm diameter, where single genes are at approximately nanomolar concentrations. A single gene, in a compartment of 2.6 μm diameter is at a concentration of 0.2 nM. This gene concentration is high enough for efficient translation. Compartmentalisation in such a volume also ensures that even if only a single molecule of the gene product is formed it is present at about 0.2 nM, which is important if the gene product is to have a modifying activity of the nucleotide sequence itself. The volume of the microcapsule should thus be selected bearing in mind not only the requirements for transcription and translation of the nucleotide sequence, but also the modifying activity required of the gene product in the method of the invention.

The size of emulsion microcapsules may be varied simply by tailoring the emulsion conditions used to form the emulsion according to requirements of the selection system. The larger the microcapsule size, the larger is the volume that will be required to encapsulate a given nucleotide sequence library, since the ultimately limiting factor will be the size of the microcapsule and thus the number of microcapsules possible per unit volume.

The size of the microcapsules is selected not only having regard to the requirements of the transcription/translation system, but also those of the selection system employed for the nucleotide sequence. Thus, the components of the selection system, such as a chemical modification system, may require reaction volumes and/or reagent concentrations which are not optimal for transcription/translation. As set forth herein, such requirements may be accommodated by a secondary re-encapsulation step; moreover, they may be accommodated by selecting the microcapsule size in order to maximise transcription/translation and selection as a whole. Empirical determination of optimal microcapsule volume and reagent concentration, for example as set forth herein, is preferred.

Preferably, PCR is used to assemble the library, introduce mutations and to amplify the selected genetic elements.

Isolating/Sorting/Selecting

The terms “isolating”, “sorting” and “selecting”, as well as variations thereof, are used herein.

“Isolation”, according to the present invention, refers to the process of separating an polypeptide domain with a desired specificity from a population of polypeptide domains having a different specificity.

In a preferred embodiment, isolation refers to purification of an polypeptide domain essentially to homogeneity.

“Sorting” of a polypeptide domain refers to the process of preferentially isolating desired polypeptide domains over undesired polypeptide domains. In as far as this relates to isolation of the desired polypeptide domains, the terms “isolating” and “sorting” are equivalent. The method of the present invention permits the sorting of desired nucleotide sequences from pools (libraries or repertoires) of nucleotide sequences which contain the desired nucleotide sequence.

“Selecting” is used to refer to the process (including the sorting process) of isolating a polypeptide domain according to a particular property thereof.

In a highly preferred application, the method of the present invention is useful for sorting libraries of polypeptide (e.g. antibody) domain nucleotide sequences. The invention accordingly provides a method, wherein the polypeptide domain nucleotide sequences are isolated from a library of nucleotide sequences encoding a repertoire of polypeptide domains, for example, antibody domains. Herein, the terms “library”, “repertoire” and “pool” are used according to their ordinary signification in the art, such that a library of nucleotide sequences encode a repertoire of gene products. In general, libraries are constructed from pools of nucleotide sequences and have properties, which facilitate sorting.

Method of In Vitro Evolution

According to a further aspect of the present invention, therefore, there is provided a method of in vitro evolution comprising the steps of: (a) selecting one or more polypeptide domains from a library according to the present invention; (b) mutating the selected polypeptide domain(s) in order to generate a further library of nucleotide sequences encoding a repertoire of gene products; and (c) iteratively repeating steps (a) and (b) in order to obtain a polypeptide domain with enhanced specificity.

Mutations may be introduced into the nucleotide sequences using various methods that are familiar to a person skilled in the art—such as the polymerase chain reaction (PCR). PCR used for the amplification of DNA sequences between rounds of selection is known to introduce, for example, point mutations, deletions, insertions and recombinations.

In a preferred aspect, the invention permits the identification and isolation of clinically or industrially useful polypeptide domains. In a further aspect of the invention, there is provided a polypeptide domain when isolated, obtained or obtainable by the method of the invention.

The selection of suitable encapsulation conditions is desirable. Depending on the complexity and size of the library to be screened, it may be beneficial to set up the encapsulation procedure such that 1 or less than 1 nucleotide sequence is encapsulated per microcapsule. This will provide the greatest power of resolution. Where the library is larger and/or more complex, however, this may be impracticable; it may be preferable to encapsulate nucleotide sequences together and rely on repeated application of the method of the invention to achieve sorting of the desired activity. A combination of encapsulation procedures may be used to obtain the desired enrichment.

Theoretical studies indicate that the larger the number of nucleotide sequence variants created the more likely it is that a molecule will be created with the properties desired (see Perelson and Oster, 1979 for a description of how this applies to repertoires of antibodies). Recently it has also been confirmed practically that larger phage-antibody repertoires do indeed give rise to more antibodies with better binding affinities than smaller repertoires (Griffiths et al., 1994). To ensure that rare variants are generated and thus are capable of being selected, a large library size is desirable. Thus, the use of optimally small microcapsules is beneficial.

In addition to the nucleotide sequences described above, the artificial microcapsules will comprise further components required for the sorting process to take place. Other components of the system will for example comprise those necessary for transcription and/or translation of the nucleotide sequence. These are selected for the requirements of a specific system from the following; a suitable buffer, an in vitro transcription/replication system and/or an in vitro translation system containing all the necessary ingredients, enzymes and cofactors, RNA polymerase, nucleotides, nucleic acids (natural or synthetic), transfer RNAs, ribosomes and amino acids, to allow selection of the modified gene product.

A suitable buffer will be one in which all of the desired components of the biological system are active and will therefore depend upon the requirements of each specific reaction system. Buffers suitable for biological and/or chemical reactions are known in the art and recipes provided in various laboratory texts, such as Sambrook et al., 1989.

The in vitro translation system will usually comprise a cell extract, typically from bacteria (Zubay, 1973; Zubay, 1980; Lesley et al., 1991; Lesley, 1995), rabbit reticulocytes (Pelham and Jackson, 1976), or wheat germ (Anderson et al., 1983). Many suitable systems are commercially available (for example from Promega) including some which will allow coupled transcription/translation (all the bacterial systems and the reticulocyte and wheat germ TNT™ extract systems from Promega). The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected (Ellman et al., 1991; Benner, 1994; Mendel et al., 1995).

In a preferred embodiment, the in vitro transcription reaction is performed for 1 hour or less at room temperature.

After each round of selection the enrichment of the pool of nucleotide sequences for those encoding the molecules of interest can be assayed by non-compartmentalised in vitro transcription/replication or coupled transcription-translation reactions. The selected pool is cloned into a suitable plasmid vector and RNA or recombinant protein is produced from the individual clones for further purification and assay.

The invention moreover relates to a method for producing a polypeptide domain, once a nucleotide sequence encoding the gene product has been sorted by the method of the invention. Clearly, the nucleotide sequence itself may be directly expressed by conventional means to produce the polypeptide domain. However, alternative techniques may be employed, as will be apparent to those skilled in the art. For example, the genetic information incorporated in the polypeptide domain may be incorporated into a suitable expression vector, and expressed therefrom.

The invention also describes the use of conventional screening techniques to identify compounds which are capable of interacting with the polypeptide domains identified by the invention. In preferred embodiments, a polypeptide domain encoding nucleic acid is incorporated into a vector, and introduced into suitable host cells to produce transformed cell lines that express the polypeptide domain. The resulting cell lines can then be produced for reproducible qualitative and/or quantitative analysis of the effect(s) of potential drugs affecting polypeptide domain specificity. Thus polypeptide domain expressing cells may be employed for the identification of compounds, particularly small molecular weight compounds, which modulate the function of the polypeptide domains. Thus, host cells expressing polypeptide domains are useful for drug screening and it is a further object of the present invention to provide a method for identifying compounds which modulate the activity of the polypeptide domain, said method comprising exposing cells containing heterologous DNA encoding polypeptide domains, wherein said cells produce functional polypeptide domains, to at least one compound or mixture of compounds or signal whose ability to modulate the activity of said polypeptide domain is sought to be determined, and thereafter monitoring said cells for changes caused by said modulation. Such an assay enables the identification of modulators, such as agonists, antagonists and allosteric modulators, of the polypeptide domain. As used herein, a compound or signal that modulates the activity of a polypeptide domain refers to a compound that alters the specificity of the polypeptide domain in such a way that the activity of the polypeptide domain is different in the presence of the compound or signal (as compared to the absence of said compound or signal).

Cell-based screening assays can be designed by constructing cell lines in which the expression of a reporter protein, i.e. an easily assayable protein, such as β galactosidase, chloramphenicol acetyltransferase (CAT) or luciferase, is dependent on the polypeptide domain. Such an assay enables the detection of compounds that directly modulate the polypeptide domain specificity, such as compounds that antagonise polypeptide domains, or compounds that inhibit or potentiate other cellular functions required for the activity of the polypeptide domains.

The present invention also provides a method to exogenously affect polypeptide domain dependent processes occurring in cells. Recombinant polypeptide domain producing host cells, e.g. mammalian cells, can be contacted with a test compound, and the modulating effect(s) thereof can then be evaluated by comparing the polypeptide domain-mediated response in the presence and absence of test compound, or relating the polypeptide domain-mediated response of test cells, or control cells (i.e., cells that do not express polypeptide domains), to the presence of the compound.

Selection Procedure

In accordance with the present invention, only polypeptide domains that can associate with the encoding DNA are selected thus allowing the establishment of a phenotype-genotype link between the gene product and the encoding gene. The nucleotide sequence will thus comprise a nucleic acid encoding a polypeptide domain linked to the polypeptide domain gene product. Thus, in the context of the present invention, the nucleotide sequence will comprise a nucleic acid encoding a polypeptide domain linked to the polypeptide domain via an association between the DNA binding site—such as a Ter operator—and the Tus DNA binding domain.

Since the polypeptide domain-Tus DNA binding domain gene product has affinity for the DNA binding site, the Tus DNA binding domain gene product will bind to the DNA binding site and become physically linked to the nucleotide sequence which is covalently linked to its encoding sequence.

At the end of the reaction, all of the microcapsules are combined, and all nucleotide sequences and gene products are pooled together in one environment. Nucleotide sequences encoding polypeptide (e.g. antibody) domains that exhibit the desired binding—such as the native binding can be selected by various methods in the art—such as affinity purification using a molecule that specifically binds to, or reacts specifically with, the polypeptide domain.

Sorting by affinity is dependent on the presence of two members of a binding pair in such conditions that binding may occur.

In accordance with the present invention, binding pairs that may be used in the present invention include an antigen capable of binding specifically to the polypeptide (e.g. antibody) domain. The antigen may be a polypeptide, protein, nucleic acid or other molecule.

The term “binding specifically” means that the interaction between the polypeptide (e.g. antibody) domain and the antigen are specific, that is, in the event that a number of molecules are presented to the polypeptide domain, the latter will only bind to one or a few of those molecules presented. Advantageously, the polypeptide domain-antigen interaction will be of high affinity.

Using affinity purification, a solid phase immunoabsorbent is used—such as an antigen covalently coupled to an inert support (e.g. cross linked dextran beads). The immunoabsorbent is placed in a column and the polypeptide domain is run in. Antibody to the antigen binds to the column while unbound antibody washes through. In the second step, the column is eluted to obtain the bound antibody using a suitable elution buffer, which dissociates the antigen-antibody bound.

Suitably, streptavidin-coated paramagnetic microbeads (e.g. Dynabeads, Dynal, Norway), coated with biotinylated target protein, are used as the solid phase support to capture those protein-DNA complexes which display desired activity.

Various immunoabsorbents for affinity purification are known in the art, for example, protein A, protein L, protein G.

Preferably, for model selection purposes, the immunoabsorbent is protein L.

Protein L exhibits a unique combination of species-specific, immunoglobulin-binding characteristics and high affinity for many classes of antibodies and antibody fragments. Protein L is a recombinant form of a Peptostreptococcus magnus cell wall protein that binds immunoglobulins (Ig) through light-chain interactions that do not interfere with the Ig antigen-binding site. A majority of Ig sub-classes, including IgG, IgM, IgA, IgD, IgE, and IgY, from human, mouse, rat, rabbit, and chicken possess light chains and can thus be bound with high affinity by Protein L. Protein L also binds Ig fragments, including scFv and Fab.

Commercially available kits can be obtained from, for example, Clonetech and SigmaAldrich.

Polypeptide domains binding to other molecules of interest—such as proteins, haptens, oligomers and polymers—can be isolated by coating them onto the chosen solid supports instead of protein L.

Multi-Step Procedure

It will be appreciated that according to the present invention, it is not necessary for all the processes of transcription/replication and/or translation, and selection to proceed in one single step, with all reactions taking place in one microcapsule. The selection procedure may comprise two or more steps.

First, transcription/replication and/or translation of each nucleotide sequence of a nucleotide sequence library may take place in a first microcapsule. Each polypeptide domain is then linked to the nucleotide sequence, which encoded it (which resides in the same microcapsule). The microcapsules are then broken, and the nucleotide sequences attached to their respective polypeptide domains are optionally purified. Alternatively, nucleotide sequences can be attached to their respective gene products using methods which do not rely on encapsulation. For example phage display (Smith, G. P., 1985), polysome display (Mattheakkis et al., 1994), RNA-peptide fusion (Roberts and Szostak, 1997) or lac repressor peptide fusion (Cull, et al., 1992).

In the second step of the procedure, each purified nucleotide sequence attached to its polypeptide domain is put into a second microcapsule containing components of the reaction to be selected. This reaction is then initiated. After completion of the reactions, the microcapsules are again broken and the modified nucleotide sequences are selected. In the case of complicated multistep reactions in which many individual components and reaction steps are involved, one or more intervening steps may be performed between the initial step of creation and linking of polypeptide domain to nucleotide sequence, and the final step of generating the selectable change in the nucleotide sequence.

Amplification

According to a further aspect of the present invention, the method comprises the further step of amplifying the nucleotide sequences bound to the immunosorbent. Selective amplification may be used as a means to enrich for nucleotide sequences encoding the desired polypeptide domain.

In all the above configurations, genetic material comprised in the nucleotide sequences may be amplified and the process repeated in iterative steps. Amplification may be by the polymerase chain reaction (Saiki et al., 1988) or by using one of a variety of other gene amplification techniques including; Qβ replicase amplification (Cahill, Foster and Mahan, 1991; Chetverin and Spirin, 1995; Katanaev, Kurnasov and Spirin, 1995); the ligase chain reaction (LCR) (Landegren et al., 1988; Barany, 1991); the self-sustained sequence replication system (Fahy, Kwoh and Gingeras, 1991) and strand displacement amplification (Walker et al., 1992).

Preferably, amplification is performed with PCR. More preferably, amplification is performed with PCR using the forward primer OA16 (SEQ ID No. 25) and the reverse primers OA 17n (SEQ ID No. 26).

Typically the amplification comprises an initial denaturation at 94° C. for 2 min, followed by 30 cycles of denaturation at 94° C. for 15 sec, annealing at 72° C. for 30 sec, extension at 72° C. for 30 sec and a final extension at 72° C. for 5 min.

Construct

The term “construct”—which is synonymous with terms such as “conjugate”, “cassette” and “hybrid”—includes a nucleic acid sequence directly or indirectly attached to a promoter. An example of an indirect attachment is the provision of a suitable spacer group such as an intron sequence, intermediate the promoter and the nucleotide sequence. The same is true for the term “fused” in relation to the present invention, which includes direct or indirect attachment.

Preferably, the promoter is a T7 promoter. More preferably, the T7 promoter is upstream of the nucleotide sequence.

The construct may even contain or express a marker, which allows for the selection of the construct in, for example, a bacterium.

Vectors

The nucleotide sequences of the present invention may be present in a vector.

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an E. coli plasmid to a bacterium, such as of the genus Bacillus, then the transformation vector is sometimes called a “shuttle vector”. It may even be a construct capable of being transferred from an E. coli plasmid to an Agrobacterium to a plant. The vectors may be transformed into a suitable host cell to provide for expression of a polypeptide.

The vectors may be for example, plasmid, virus or phage vectors provided with an origin of replication, optionally a promoter for the expression of the said polynucleotide and optionally a regulator of the promoter.

The vectors may contain one or more selectable marker nucleotide sequences. The most suitable selection systems for industrial micro-organisms are those formed by the group of selection markers which do not require a mutation in the host organism. Examples of fungal selection markers are the nucleotide sequences for acetamidase (amdS), ATP synthetase, subunit 9 (oliC), orotidine-5′-phosphate-decarboxylase (pvrA), phleomycin and benomyl resistance (benA). Examples of non-fungal selection markers are the bacteria G418 resistance nucleotide sequence (this may also be used in yeast, but not in filamentous fungi), the ampicillin resistance nucleotide sequence (E. coli), the neomycin resistance nucleotide sequence (Bacillus) and the E. coli uidA nucleotide sequence, coding for β-glucuronidase (GUS).

Vectors may be used in vitro, for example for the production of RNA or used to transfect or transform a host cell.

Thus, polynucleotides may be incorporated into a recombinant vector (typically a replicable vector), for example a cloning or expression vector. The vector may be used to replicate the nucleic acid in a compatible host cell.

Genetically engineered host cells may be used for expressing an amino acid sequence (or variant, homologue, fragment or derivative thereof).

Expression Vectors

The nucleotide sequences of the present invention may be incorporated into a recombinant replicable vector. The vector may be used to replicate and express the nucleotide sequence in and/or from a compatible host cell. Expression may be controlled using control sequences, which include promoters/enhancers and other expression regulation signals. Prokaryotic promoters and promoters functional in eukaryotic cells may be used. Chimeric promoters may also be used comprising sequence elements from two or more different promoters described above.

The protein produced by a host recombinant cell by expression of the nucleotide sequence may be secreted or may be contained intracellularly depending on the sequence and/or the vector used. The coding sequences can be designed with signal sequences, which direct secretion of the substance coding sequences through a particular prokaryotic or eukaryotic cell membrane.

Fusion Proteins

Amino acid sequences of the present invention may be produced as a fusion protein, for example to aid in extraction and purification, using a tag sequence.

Host Cells

As used herein, the term “host cell” refers to any cell that may comprise the nucleotide sequence of the present invention and may be used to express the nucleotide sequence.

Thus, in a further embodiment the present invention provides host cells transformed or transfected with a polynucleotide that is or expresses the nucleotide sequence of the present invention. Preferably, said polynucleotide is carried in a vector for the replication and expression of polynucleotides. The cells will be chosen to be compatible with the said vector and may for example be prokaryotic (for example bacterial), fungal, yeast or plant cells.

The gram-negative bacterium E. coli is widely used as a host for heterologous nucleotide sequence expression. However, large amounts of heterologous protein tend to accumulate inside the cell. Subsequent purification of the desired protein from the bulk of E. coli intracellular proteins can sometimes be difficult.

In contrast to E. Coli, bacteria from the genus Bacillus are very suitable as heterologous hosts because of their capability to secrete proteins into the culture medium. Other bacteria suitable as hosts are those from the nucleotide sequencera Streptomyces and Pseudomonas.

Depending on the nature of the polynucleotide and/or the desirability for further processing of the expressed protein, eukaryotic hosts such as yeasts or other fungi may be preferred.

The use of host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Regulatory Sequences

In some applications, polynucleotides may be linked to a regulatory sequence, which is capable of providing for the expression of the nucleotide sequence, such as by a chosen host cell. By way of example, the present invention covers a vector comprising the nucleotide sequence of the present invention operably linked to such a regulatory sequence, i.e. the vector is an expression vector.

The term “regulatory sequences” includes promoters and enhancers and other expression regulation signals.

The term “promoter” is used in the normal sense of the art, e.g. an RNA polymerase binding site.

Enhanced expression of polypeptides may be achieved by the selection of heterologous regulatory regions, e.g. promoter, secretion leader and terminator regions, which serve to increase expression and, if desired, secretion levels of the protein of interest from the chosen expression host and/or to provide for the inducible control of expression.

Aside from the promoter native to the nucleotide sequence encoding the polypeptide, other promoters may be used to direct expression of the polypeptide. The promoter may be selected for its efficiency in directing the expression of the polypeptide in the desired expression host.

In another embodiment, a constitutive promoter may be selected to direct the expression of the polypeptide. Such an expression construct may provide additional advantages since it circumvents the need to culture the expression hosts on a medium containing an inducing substrate.

Examples of strong constitutive and/or inducible promoters which are preferred for use in fungal expression hosts are those which are obtainable from the fungal nucleotide sequences for xylanase (xlnA), phytase, ATP-synthetase, subunit 9 (oliC), triose phosphate isomerase (tpi), alcohol dehydrogenase (AdhA), α-amylase (amy), amyloglucosidase (AG—from the glaA nucleotide sequence), acetamidase (amdS) and glyceraldehyde-3-phosphate dehydrogenase (gpd) promoters.

Examples of strong yeast promoters are those obtainable from the nucleotide sequences for alcohol dehydrogenase, lactase, 3-phosphoglycerate kinase and triosephosphate isomerase.

Examples of strong bacterial promoters are the α-amylase and SP02 promoters as well as promoters from extracellular protease nucleotide sequences.

Hybrid promoters may also be used to improve inducible regulation of the expression construct.

The promoter can additionally include features to ensure or to increase expression in a suitable host. For example, the features can be conserved regions such as a Pribnow Box, a TATA box or T7 transcription terminator. The promoter may even contain other sequences to affect (such as to maintain, enhance, decrease) the levels of expression of a nucleotide sequence. Suitable other sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat Gene 217 [1987] 217-225; and Dawson Plant Mol. Biol. 23 [1993] 97).

If the nucleotide sequence comprises a regulatory sequence, then in one embodiment, the regulatory sequence may be located in between the one or more DNA binding sites and one or more polypeptide domains.

If the nucleotide sequence comprises a regulatory sequence, then in a further embodiment, the regulatory sequence may be located upstream of the one or more DNA binding sites, and downstream of the one or more polypeptide domains and one or more Tus DNA binding domains.

Variants/Homologues/Derivatives

The present invention encompasses the use of variants, homologues, derivatives and/or fragments of the nucleotide and/or amino acid sequences described herein.

The term “variant” is used to mean a naturally occurring polypeptide or nucleotide sequences which differs from a wild-type sequence.

The term “fragment” indicates that a polypeptide or nucleotide sequence comprises a fraction of a wild-type sequence. It may comprise one or more large contiguous sections of sequence or a plurality of small sections. The sequence may also comprise other elements of sequence, for example, it may be a fusion protein with another protein. Preferably the sequence comprises at least 50%, more preferably at least 65%, more preferably at least 80%, most preferably at least 90% of the wild-type sequence.

The term “homologue” means an entity having a certain homology with the subject amino acid sequences and the subject nucleotide sequences. Here, the term “homology” can be equated with “identity”.

In the present context, a homologous sequence is taken to include an amino acid sequence, which may be at least 70, 75, 80, 85 or 90% identical, preferably at least 95, 96, 97, 98 or 99% identical to the subject sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence, which may be at least 70, 75, 80, 85 or 90% identical, preferably at least 95, 96, 97, 98 or 99% identical to the subject sequence.

Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix—such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example, according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution—such as basic for basic, acidic for acidic, polar for polar etc. Non-homologous substitution may also occur i.e. from one class of residue to another or alternatively involving the inclusion of unnatural amino acids—such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids—such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe)—such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminoproprionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups—such as methyl, ethyl or propyl groups—in addition to amino acid spacers—such as glycine or β-alanine residues. A further form of variation involves the presence of one or more amino acid residues in peptoid form will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example, Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences may be modified by any method available in the art. Such modifications may be carried out to enhance the in vivo activity or life span of nucleotide sequences useful in the present invention.

The present invention may also involve the use of nucleotide sequences that are complementary to the nucleotide sequences or any derivative, fragment or derivative thereof. If the sequence is complementary to a fragment thereof then that sequence can be used as a probe to identify similar coding sequences in other organisms etc.

Preferably, the resultant nucleotide sequence encodes an amino acid sequence that has the same activity. The resultant nucleotide sequence may encode an amino acid sequence that has the same activity, but not necessarily the same degree of activity.

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The invention will now be further described by way of Examples, which are meant to serve to assist one of ordinary skill in the art in carrying out the invention and are not intended in any way to limit the scope of the invention.

### EXAMPLES

### Example 1

### Construction and Expression of Constructs Used

pIE2

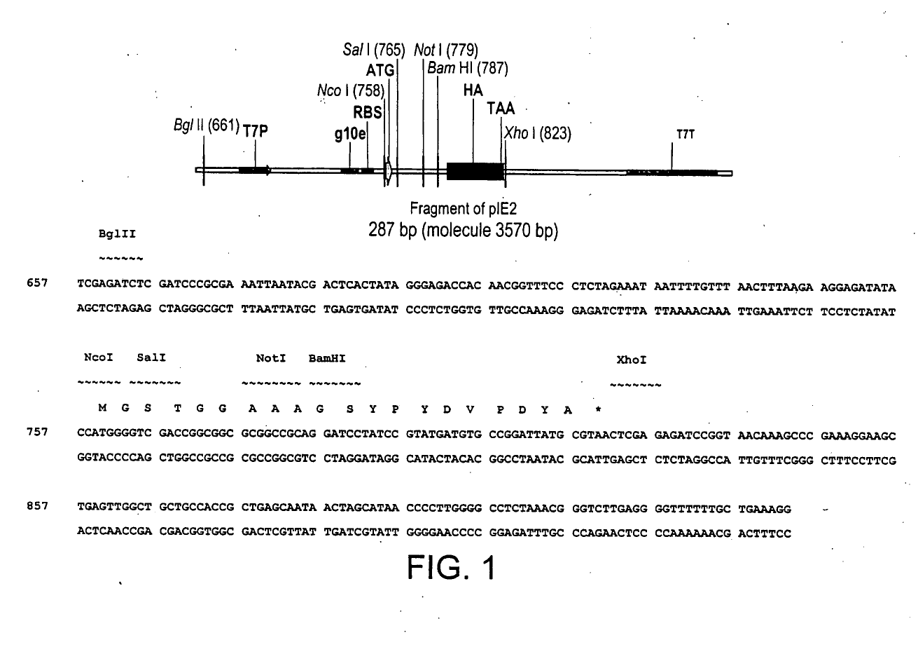

Genetic elements for the in vitro expression of domain antibodies in fusion to the N-terminus of Tus are based on the pIE2 in vitro expression vector (FIG. 1). pIE2 is assembled by ligating the DNA duplex formed from the annealed phosphorylated oligonucleotides AS5 (SEQ ID No. 10) and AS6 (SEQ ID No. 11) into the gel purified Nco I/Not I—cut pIE1 vector. pIE1 is assembled by ligating the DNA duplex formed from the annealed phosphorylated oligonucleotides AS1 (SEQ ID No. 12) and AS2 (SEQ ID No. 13) is into gel purified NcoI/BamHI-cut pIVEX2.2b Nde (Roche) in vitro expression vector. Typically both oligonucleotides used in a reaction are phosphorylated simultaneously in 50 μl volume at 2 μM concentration using 5 units of T4 polynucleotide kinase (NEB) in T4 DNA ligase buffer (NEB). Polynucleotide kinase is inactivated by 5 min incubation of the reaction mix at 95° C., followed by 30 min cooling step to 40° C. to allow the annealing of the oligonucleotides to take place. 0.1 μl aliquot of the annealed phosphorylated DNA duplex is added to 100 ng of digested and phosphorylated vector and ligated for 1 h at room temperature in 5 μl volume using 50 units of T4 DNA ligase (NEB). 0.5 μl aliquots of the ligation reaction are thereafter used to transform 5 μl aliquots of supercompetent XL-10 E. coli cells (Stratagene) according to the manufacturer's instructions. The sequences of the inserted fragments are verified by DNA sequencing of plasmid DNA minipreps (Qiagen) prepared from overnight cultures.

Tus Containing Constructs

Tus was PCR amplified from E. coli TG1 genomic DNA using SuperTaq DNA polymerase with primers AS102 (SEQ ID No. 14) and AS103 (SEQ ID No. 15). The product was cleaned and digested with the restriction enzymes BamH I and Bgl II (NEB). The digested product was ligated into the BamH I site of pIE2 to yield pIE2T. The construct was verified by DNA sequencing.

The following in vitro expression constructs with TerB operator sites are used.

pIE2tT construct is based on the pIE2T vector, with one TerB operator site inserted into a unique Bgl II-site just upstream of the T7 promoter. The TerB operator motif was assembled from annealed, phosphorylated oligonucleotides AS105 (SEQ ID No. 16) and AS114 (SEQ ID No. 17) and ligated into Bgl II-cut, CIAP-dephosphorylated pIE2T vector.

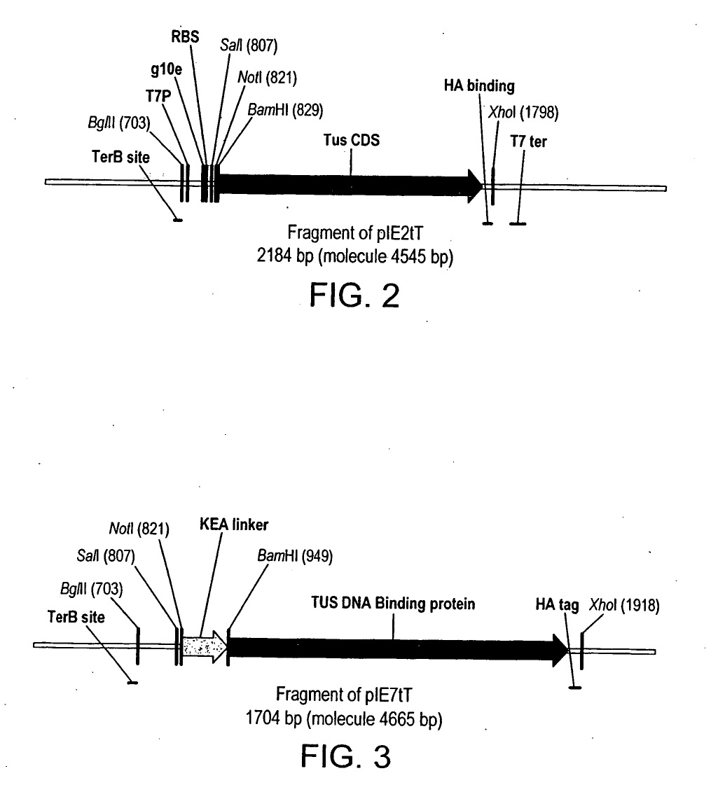

A clone sequenced with primer AS16 (SEQ ID No. 18), where the insert orientation leaves Bgl II site upstream of the TerB operator insert, i.e. closer to T7 promoter, is adopted for future work (FIG. 2). More TerB operator sites can be inserted into the vectors by cutting the construct with Bgl II and inserting the next copy of the operator site, assembled from the annealed phosphorylated oligonucleotides AS105 (SEQ ID No. 16)/AS114 (SEQ ID No. 17).

Insertion of (KEA₃)₈ Linker in pIE2tT

pIE7'tT was obtained by cutting the Not I site of pIE2tT and inserting AS120 (SEQ ID No. 19)-AS121 (SEQ ID No. 20) kinased duplex. Subsequently, pIE7tT was obtained by cutting the Not I site of pIE7'tT and repeating the insertion of AS120 (SEQ ID No. 19)-AS121 (SEQ ID No. 20) kinased duplex (FIG. 3).

Tus Fusion Constructs with V_{κ}-Domain Antibody (Dab)

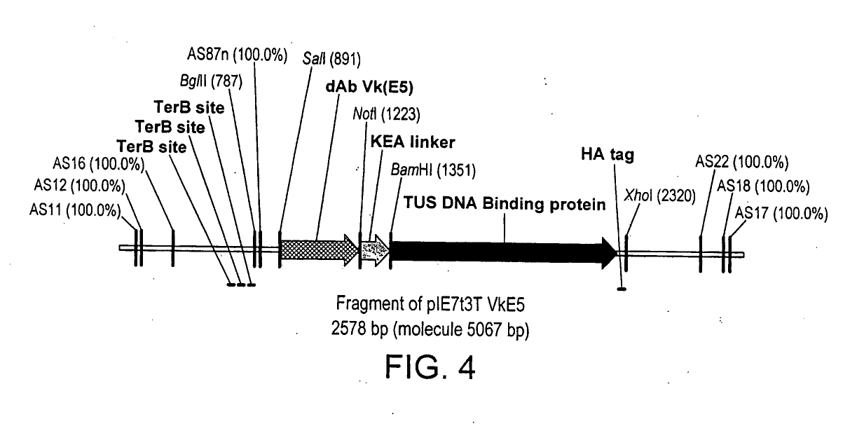

Anti-β-galactosidase Vₖ clone E5, TNFa binding Vₖ clones TAR1-5-19 and TAR1-5, and cytokine A binding Vₖ clone X can all be cloned into Sal I/Not I cut pIE7t³T vector already harbouring the Tus construct and three Ter-B operators. As an example, fusion construct of Vₖ(E5) (SEQ ID No. 7) to the N-terminus of Tus (pIE7t³T-series) is shown in FIG. 4 with three TerB operator sites inserted into the Bgl II site, yielding construct pIE7t³T.Vₖ(E5).

It can be expected that more than one in vitro expressed Vₖ(E5)-Tus molecule will bind the genetic element within the compartment if the number of TerB operator sites is increased, leading potentially to a more stable genotype—phenotype linkage. Therefore, the expression constructs with Vₖ(E5) (SEQ ID No. 7) fused to the N-terminus of Tus were prepared harbouring also two, three and four copies of TerB operator, allowing up to tetravalent interaction with the DNA. The distance between the operator sites was chosen to be 19 bp, corresponding approximately to the one-and-half helical turns of the DNA helix, ensuring that all bound Vₖ moieties of the bound Vₖ-Tus fusion protein would be exposed in opposite directions, limiting simultaneous multivalent contact with any soluble target molecules.

### Example 2

### Functionality of Domain Antibody Unaffected by Fusion to Tus

To isolate domain antibodies that bind specifically a given antigen, it is preferable that the domain antibody functions similarly when fused to Tus as when functioning as a monomer in solution.

Fusion constructs were made of Vₖ(TAR1-5-19) (SEQ ID No. 5) or Vₖ(E5) (SEQ ID No. 7) fused to the N-terminus of Tus through either a short A₃GS linker or a long, rigid α-helical linker (KEA₃)₈. Both Vₖ'S were digested SalI-NotI and ligated in vector pIE2tT or pIE7tT, respectively, which had also been digested SalI-NotI. The ligation mixture was transformed to XL-10 gold cells (Stratagene) and cells were plated. After miniprepping (Qiagen) and confirmation by DNA sequencing, the constructs were PCR amplified with primers AS11-AS17 to yield a fragment containing: one TerB operator site—T7 promoter—Vₖ(TAR1-5-19)/Vₖ(E5)—A₃GS/(KEA₃)₈— Tus—HA—T7 terminator. The typical amplification cycle for this PCR is performed with platinum pfx DNA polymerase (invitrogen) and consists of: initial denaturation of 3 min at 95 C, followed by 25 cycles of 30 seconds at 95 C, 30 seconds at 60 C, and 2 minutes at 68 C; and a final extension at 68 C for 3 minutes. The PCR product is cleaned on a Qiagen spin column, eluted and the DNA concentration determined by OD 260/280. The cleaned PCR product is used for in vitro transcription/translation (IVT). A typical 50 μl IVT reaction consists of 500 ng of DNA, 2.0 λl methionine (5 mM), 1.5 μl oxidized glutathione (100 mM) (Sigma), 35 μl bacterial extract, e.g. EcoPro (Novagen), and 11 μl H₂O. The IVT reaction can be performed for 1 up to 4 hours at temperatures between 20 C and 37 C. After IVT, the reaction is diluted 1 in 10 in PBS+0.2% tween-20. Fifty μl are added to an ELISA plate, that has been coated with anti-HA (3F10, Roche) (1 μg/ml in PBS), and incubated for 1 hour at room temperature. After washing, a concentration range (0-500 nM) of biotinylated antigen, i.e. TNFa, is added and incubated on the plate for 1 hour. Again, plate is washed and streptavidin conjugated to horse radish peroxidase (Streptavidin-HRP, Amersham) at a dilution of 1:3500 is added and incubated on the plate for 30 minutes. After a final wash, TMB substrate is added and colouring reaction is let to proceed for 15-30 min and stopped by addition of 1M HCl.

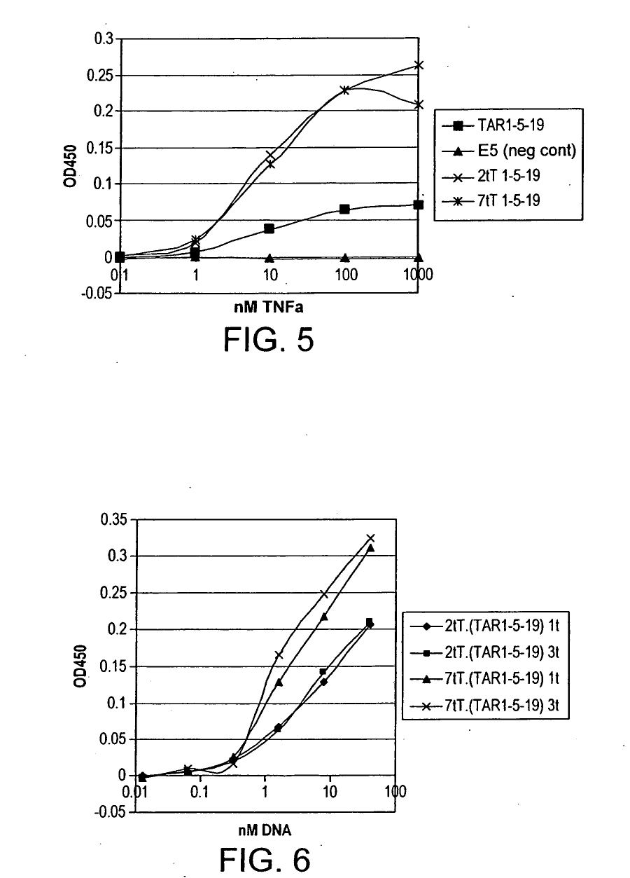

When TNFa concentration is plotted against signal (FIG. 5) the IC₅₀ can be determined by the concentration at which the half-maximal signal is obtained. Comparison of the IC₅₀-value found for Vₖ(TAR1-5-19) (SEQ ID No. 5) fused to Tus is independent of the linker used and similar to that determined for Vₖ(TAR1-5-19) (SEQ ID No. 5) as a monomeric domain antibody in solution.

This result demonstrates that the Vₖ(TAR1-5-19) (SEQ ID No. 5) behaves similarly when fused to Tus as when acting as a Vₖ in solution.

### Example 3

### DNA Binding Functionality of Tus is Substantially Unaffected by N-Terminal Fusion to a Domain Antibody

Suitably, the domain antibody should be substantially unaffected by fusion to Tus, and the DNA binding properties of Tus should be sufficiently retained. As already described in Example 2, where the binding affinity of the domain antibody is evaluated, the binding of Tus can be determined.

After in vitro translation of either pIE2tT.Vₖ(TAR1-5-19) or pIE7tT.Vₖ(TAR1-5-19), the fusion protein is captured on anti-HA coated ELISA plates and incubated for about one hour with either a single (1t) or triple (3t) biotinylated TerB operator(s). The biotinylated TerB operators are made by PCR amplification of the TerB operator sequence in either pIE7tT or pIE7t³T vector using the oligonucleotide pair AS92 (SEQ ID No. 27) (biotinylated) and AS87n (SEQ ID No. 28). Incubation of the captured IVT product with a concentration range (0.012-40 nM) of biotinylated operators, followed by washing, incubation with streptavidin-HRP, and colouring with TMB substrate, gives a result as seen in FIG. 6. The very high affinity for free DNA operator sequences is advantageously retained. Furthermore, Tus is functional with both linkers though preferably the KEA linker (pIE7tT-serie) is used.

For Tus to be functional during selections, Tus should bind to its corresponding DNA at least for the time of the experiment. The half-life of the DNA-Tus complex has previously been determined (Skokotas et al., (1995) J Biol. Chem. 29; 270(52):30941-8) at 149 minutes. To determine if the half-life when fused to a domain antibody is similar, the following experiment can be performed. In a 50 μl PBS/tween-20 solution, 3 μl of IVT reaction using pIE2tT.Vₖ(TAR1-5-19) or pIE7tT.Vₖ(TAR1-5-19) as template is incubated for about one hour with 15 nM of biotinylated 1t or 3t free operator. Subsequently, dAb-Tus-HA fusion protein, with or without operator bound to it, is captured for about one hour on an anti-HA coated ELISA plate. After 1-hour incubation, the plate is washed, removing unbound biotinylated operator, and replaced with 10 nM non-biotinylated (‘cold’) operator. At different time points (0-4 hours) the ‘cold’ operator is removed, the well is washed and incubated with streptavidin-HRP (dilution 1:3500). Wells are washed and incubated with TMB substrate for a fixed amount of time (e.g. 15 minutes) and the reaction is stopped by addition of 1M HCl.

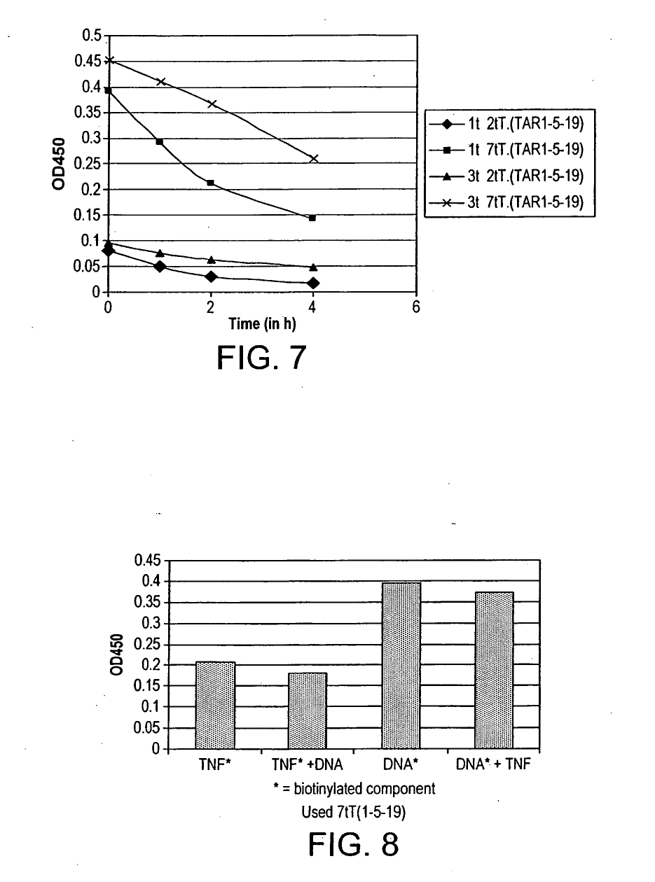

When time is plotted against signal the dissociation rate of bio-1t or 3t is determined (FIG. 7).

Both linkers work, although a preference exists for the KEA linker. The value found for the half-life of 1t bound to Tus (2.5 hours) is in agreement with reported literature values. This agreement confirms the DNA-binding functionality of Tus when fused to a domain antibody. Furthermore, the longer half-life of the 3t fragment would make it desirable to use three operators instead of one.

### Example 4

### Neither DNA Binding Functionality Nor Antigen Binding Affinity of a Vk are Affected by the Concomitant Addition of the Other Component

In the previous Examples, we demonstrated that the domain antibody recognises its antigen with similar affinity in solution as when fused to Tus. Similarly, Tus binds its TerB operator DNA with a half-life that is close to equal that of literature values, indicating no loss of functionality when fused. However, from these experiments it is unclear if both events, domain antibody binding antigen and Tus binding DNA, can function simultaneously without influencing each other. Therefore, we sought to investigate concomitant binding to Tus and dAb.

As in previous examples, pIE7tT.Vₖ(TAR1-5-19) was in vitro translated and the product diluted (1:10) in PBS/T-20. Subsequently, the fusion protein Vₖ(TAR1-5-19)-Tus-HA is captured on an ELISA plate coated with anti-HA antibody. The plate is washed and incubated with either biotinylated tNFa (600 nM) in the absence or presence of non-biotinylated operator DNA (15 nM). Conversely, biotinylated-DNA (15 nM) is incubated in the absence or presence of non-biotinylated TNFa (600 nM). After incubation with Streptavidin-HRP (1:3500) and addition of TMB substrate, the colour is developed.

FIG. 8 represents the results, which demonstrate that addition of large amounts of non-biotinylated antigen or operator DNA has virtually no influence on the binding of the biotinylated TNFa or DNA, respectively. This stresses that both domain antibody and Tus protein bind their respective targets independently and simultaneously.

### Example 5

### Stable Genotype

### Phenotype Linkage is Retained when Selections are Performed with Reactions Compartmentalised in Separate Reaction Vials

In the previous examples we have demonstrated the functionality of domain antibodies and Tus DNA binding protein when expressed as in vitro translated fusion proteins. For selections to be performed with these fusion proteins it is however crucial that the genotype—phenotype linkage, the binding of dAb-Tus fusion protein to its corresponding DNA, is retained in solution for the time of selection. To that end, model selections can be performed between two dAbs of known but different affinities, e.g. Vₖ(TAR1-5-19) (SEQ ID No. 5) (Kd 50 nM) and Vₖ(TAR1-5) (SEQ ID No. 6) (>5 μM). By inserting a small, non-interacting DNA stuffer fragment (z³, 150 bp) in the BglII site between the TerB operator and the T7 promoter, the DNA of each dAb can have a specific length, making it possible to identify rapidly the dAb by the size of the PCR product of this region. The following constructs were used: 7t³T.Vₖ(TAR1-5) and 7t³z³T.Vₖ(TAR1-5-19). Each construct was PCR amplified with primers AS11 (SEQ ID No.21) and AS17 (SEQ ID No. 23) to obtain the PCR fragment needed for in vitro transcription/translation. In separate reaction vials each PCR fragment was translated. The typical reaction mixture is similar to that described in Example 2, however, the DNA concentration is lower, only 150 ng per 50 μl reaction, and biotinylated TNFa is present during IVT at 20 nM. The reaction mixture is incubated for 1 hour at room temperature. Both extracts are diluted 1 in 16 in PBS/T-20/bio-TNFa (20 nM) and subsequently mixed in e.g. in a 1:100 and 1:1 ratio (TAR1-5-19:TAR1-5). Fifty μl of this reaction mixture is transferred to streptavidin coated PCR tubes (Abgene) that have been blocked for 1 hour with PBS+2% Tween-20. The incubation in these wells is for 45 minutes, after which the wells are washed (PBS+T-20) and PCR with the oligonucleotide pair AS12 (SEQ ID No. 22) and AS87n (SEQ ID No. 28) is performed to amplify the stuffer fragment that differentiates the DNA templates for TAR1-5-19 and TAR 1-5. The PCR is performed using platinum pfx DNA polymerase and 30 cycles (melt 30 s at 95 C, anneal 45 s at 60 C, extend 1 min at 68 C).

The result is shown in FIG. 8 and demonstrates that at a 1:100 ratio of TAR1-5-19 over TAR1-5, in a single round, efficiently isolates the DNA of the higher affinity binder over a large abundance of low affinity binder.

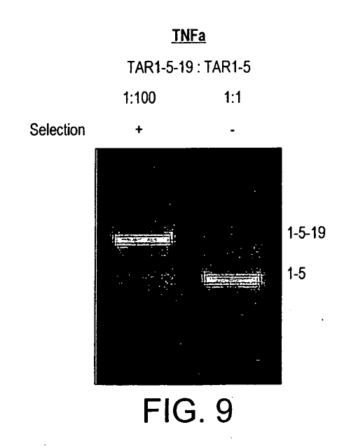

If no selection is performed and both are mixed 1:1, this 1:1 ratio is not affected (FIG. 9).

### Example 6

### Model Selection with Emulsification

In the previous Example we showed that genotype—phenotype linkage is retained when constructs are in vitro translated in separate vials prior to mixing of the translation products. When selections are to be performed using multiple templates, it is however no longer feasible to compartmentalise by performing the in vitro translation reaction for each template in a separate vial. A solution to this problem would be to perform the in vitro translation reaction in a microcapsule made by emulsifying oil in water. Each microcapsule should typically contain a single DNA template in addition to all components necessary to perform in vitro transcription/translation. After translation, the produced dAb-Tus fusion protein will bind to the DNA template present in the same microcapsule. This protein-DNA interaction should be stable enough to survive subsequent breaking of the emulsion and the selection for binding properties of the domain antibody part of the fusion protein.

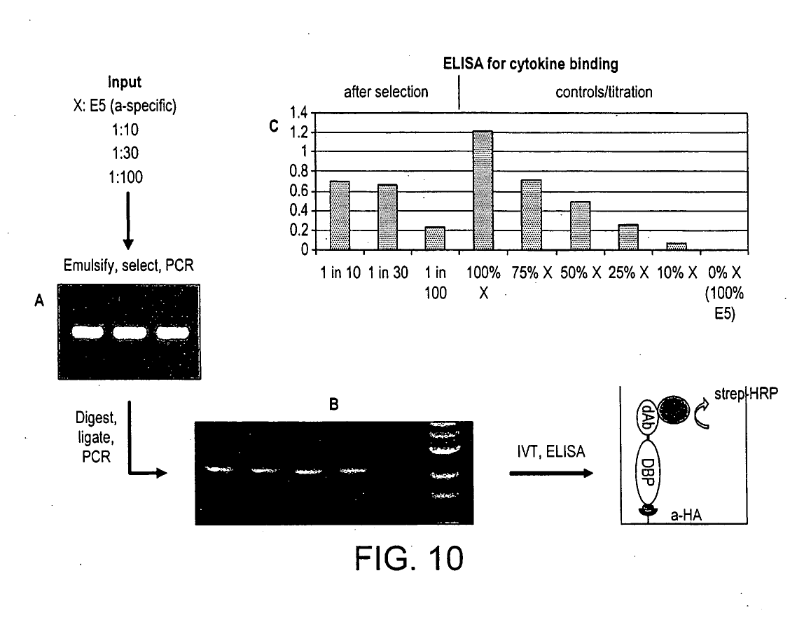

For example, two constructs 7t³T.Vₖ(X) containing a dAb that binds a cytokine with 50 nM Kd, and 7t³T.Vₖ(E5), which has no measurable affinity for the cytokine, are each PCR amplified separately with AS11 (SEQ ID No. 21) and AS17 (SEQ ID No. 23) to give linear DNA fragments consisting of three TerB operator sites-T7 promoter-dAb-linker-Tus-HA-stop (FIG. 4). These PCR products are cleaned on a Qiagen spin column, the DNA is quantified, and mixed at molar ratios 1:10, 1:30, and 1:100 (X:E5). Subsequently, in vitro translation is performed in emulsions. Typically, this is performed as follows: to a 10 ml falcon tube containing a magnetic stirrer, 650 μl of a mineral oil (sigma), 4.5% Span-80 (Fluka) and 0.5% triton-X-100 (Sigma) mixture is added. The tube is placed in a holder on a magnetic stirrer plate. Meanwhile, the DNA template solution is diluted to 1.2 ng/μl in TBS+2% BSA and 1 μl of this solution is added to a reaction vial. This amount corresponds to 5.0×10⁸ molecules of DNA. In addition to the previously mentioned components of the IVT reaction (11.5 μl H₂O, 1.5 μl oxidised glutathione, 2.0 μl methionine and 35 μl EcoPro), 10 nM of biotinylated cytokine A is added. The IVT reaction mixture is added to the DNA, mixed swiftly, and immediately added to the stirring oil. After 5 minutes of stirring, a homogenous emulsion has been created and the mixture is removed from the stirrer and incubated at room temperature for 1 hour. Subsequently, the emulsion is broken. This is performed by adding the emulsion to 250 μl PBS/1% BSA, containing biotinylated cytokine A (10 nM), and 0.5 ml of hexane/mineral oil (80/20). The mix is vortexed and centrifuged for 1 min at 13000 rpm, the organic top layer is removed, and 1 ml of hexane/MO is added. This procedure is repeated 3 times. The fourth time, only hexane is added and removed after centrifugation. The water phase is transferred to streptavidin coated PCR tubes (ABgene) and incubated for 30 minutes followed by washing with PBS/1% BSA. Fifty μl of PCR reaction mixture, containing primers OA16 (SEQ ID No. 25), OA17n (SEQ ID No. 26) and pfuUltra DNA polymerase (Stratagene), is added to the tubes. Subsequently, 30 cycles of amplification is performed using the following conditions: melt at 95 C for 30s, anneal and amplify at 72 C for 30s. The PCR product is checked on a 2% agarose gel (FIG. 10) and cleaned on a Qiagen spin column. The product is digested with the restriction enzymes SalI and NotI (NEB) in 50 μl and ligated in the pIE7t³T vector that had also been digested SalI-NotI. The ligation is performed using T4 ligase (NEB) in a total volume of 5 μl. One μl of the ligation reaction is PCR amplified in 25 cycles with primers AS16 (SEQ ID No. 18) and AS22, using platinum pfx DNA polymerase. After cleaning and analysis on a 1.2% agarose gel (FIG. 10), the PCR product can subsequently be in vitro translated and analysed for antigen binding as described in Example 2. In this case, incubation with cytokine A is performed at a single concentration (100 nM) and the results are plotted (FIG. 10).

A single round of selection increases the level of binders to the cytokine by 25-fold, as is visualised when comparing e.g. the signal after selection of 1:30 (3.3%) and 1:100 (1%) to the values for titration curves at 75% and 25%, respectively.

### Example 7

### Affinity Maturation of a Cytokine-Binding Domain Antibody from a Library of Domain Antibodies

One application of the invention is the affinity maturation of a domain antibody. Frequently, one has an antibody to an antigen of a given affinity. However, this affinity is insufficient for the antibody to be e.g. therapeutically useful. Therefore, one will want to further improve the affinity of the antibody. Most approaches require the generation of a vast number of mutants of the parent antibody, followed by selection for a better binder. Using genotype—phenotype linkage with the Tus DNA binding domain in combination with in vitro transcription/translation in microcapsules would make it possible to assess diversities of 108 antibody variants for better binding properties.

An example of the use of the Tus system for affinity maturation purposes is the following: a domain antibody Y with a Kd of 10 nM for cytokine A was taken as parent. In the first step, the parent molecule, in pDOM5, was amplified with primers DOM8 (SEQ ID No. 29) and DOM9 (SEQ ID No. 30) to yield a PCR fragment containing the dAb. Subsequently, the dAb gene was PCR amplified with primers OA16 (SEQ ID No. 25) and OA17n (SEQ ID No. 26) using the GenemorphII kit (Stratagene) to create random errors in the parent sequence. The error-prone PCR was performed according to manufacturers instructions. Briefly, one pg of DOM 8-DOM 9 product was amplified for 30 cycles (melt 30s at 95 C, anneal and extend 30s at 72 C). The product was cleaned on a Qiagen column, digested with restriction enzymes SalI-NotI, cleaned again on a Qiagen spin column, and ligated using T4 DNA ligase in the pIE7t³T vector. To assess the diversity after the ligation, 0.5 μl aliquot was transformed in to XL-10 gold cells (Stratagene) and dilutions were plated. Alongside, a known amount of miniprepped DNA, 7t³T.Vk(Y), was diluted in 1×T4 ligase buffer and also transformed to XL-10 cells and plated. By counting the number of colonies on both the ligation mixture and control plates, and multiplying by the dilution rate, an estimate was made of the number of ligation events. In most cases, this number exceeded 10⁸. A few colonies were picked and sequenced to verify that diversification had occurred.

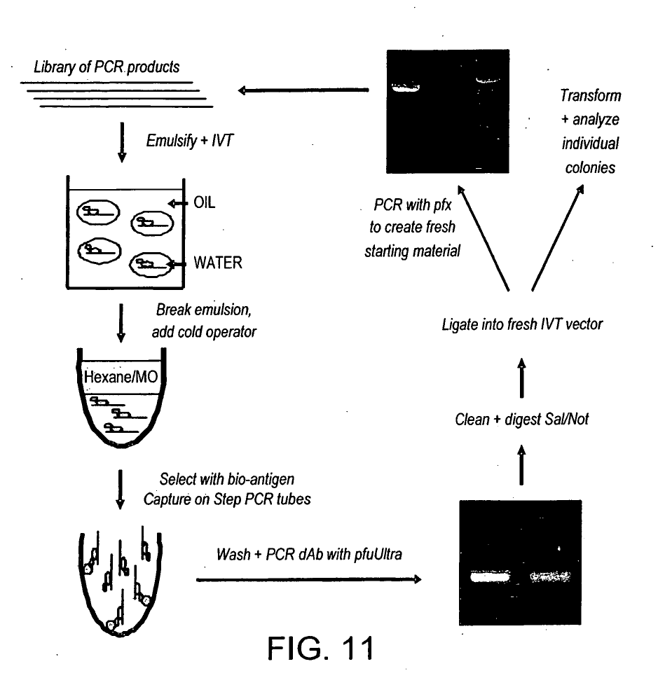

In the next step, the ligation mixture containing the error-proned gene was PCR amplified using platinum pfx DNA polymerase and primers AS12 (SEQ ID No. 22) and AS18 (SEQ ID No. 24). The PCR program used was generally: 25 cycles, met 30s at 95 C; anneal 30s at 60 C, extend 2 min 68 C. After amplification the product was checked on a 1.2% agarose gel, cleaned on a Qiagen column, and quantified by OD260/280. This PCR product was used as input material for the first round of selection. A detailed description of how a round of selection in emulsion is performed is given in example 6 and summarized in FIG. 11. In this example of affinity maturation selection a few modifications were made:
1) To the IVT reaction mixture cytokine Y was added at 50 nM concentration. This means that during in vitro transcription/translation the antigen was already present in the microcapsule in the emulsion.
2) After IVT in emulsion, the emulsion was broken in the presence of 250 μl of PBS/1% BSA. To this waterphase 2 nM of free 3t operator fragments was added to scavenge any dAb-Tus fusion protein that dissociated from its cognate DNA during and after breaking of the emulsion.
3) Also to the 250 μl of PBS/BSA used during the breaking of the emulsion, additional biotinylated antigen was added in such an amount that the final concentration remained the same as during the IVT. In the first round this was 50 nM, in subsequent rounds this was reduced to 10 nM.
4) The possibility exists to perform off-rate selections. This was done by adding non-biotinylated (‘cold’) antigen to the reaction mixture after the emulsion had been broken and prior to capture of the antigen/dAb-Tus/DNA complex on streptavidin coated PCR tubes. The length of time during which off-rate selections were performed varied as the stringency of selection conditions was increased during sequential rounds of selection. In this example, off-rate selections started in round 4, for 5 min, and increased to 20 min in round 9.

After IVT in the microcapsule, breaking of the emulsion, and capture on streptavidin coated PCR tubes (all as described in example 6), the DNA encoding the binding dAb was PCR amplified with primers OA16 (SEQ ID No. 25) and OA17n (SEQ ID No. 26). At this stage, the option is available to introduce extra mutations in the selected clones by performing an additional —PCR using error-prone conditions. This was done after three rounds of selection and similar conditions were used as previously described for the making of error-prone libraries. In all cases, the products were digested with restriction enzymes SalI and NotI, ligated in pIE7t³T and PCR amplified with oligonucleotides AS12 (SEQ ID No. 22) and AS18 (SEQ ID No. 24). The PCR product of this reaction was used for a next round of selection. In this example a total of nine sequential rounds of selection were performed. During the rounds, decreasing amounts of biotinylated antigen were used: 50 nM in round 1, 20 nM in round 2, and 10 nM in rounds 3 to 9. Off-rate selections were performed during rounds 4-9 with the following concentrations and times: round 4, 5 minutes with 400 nM cold antigen; round 5, 8 minutes with 400 μM cold antigen; round 6, 15 minutes with 600 nM cold antigen; round 7, 20 minutes with 600 nM cold antigen; round 8, 20 minutes with 1 μM cold antigen; and round 9, 20 minutes with 1 μM cold antigen;

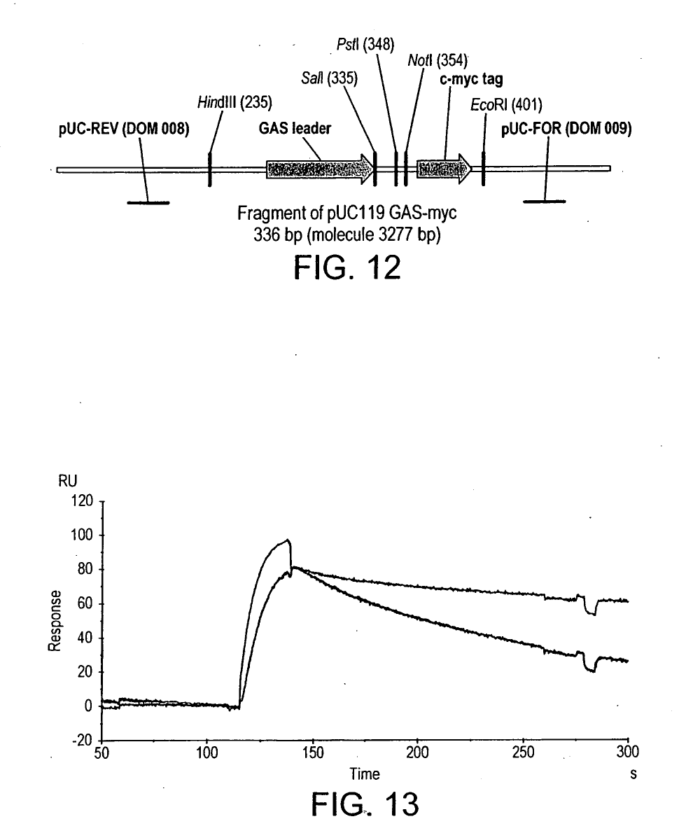

After round 9, the selected domain antibodies were cloned SalI-NotI into a pUC119 based expression vector under control of the lacZ promoter (FIG. 12), and transformed to HB2151 cells. dAbs were randomly picked, expressed, purified, and characterised. Characterisation of the affinity of the dAbs for cytokine A was performed on a BIAcore1000.

In this Example, a clone (Vₖ(X*)) characterised on the BIAcore contained three amino acid mutations and its affinity for the antigen had increased approximately 10 times (FIG. 13).

### Example 8

Affinity maturation of a Cytokine X binding domain antibody from a library of domain antibodies.

To verify that the use of our technology to affinity mature domain antibodies is not limited to a single target, we performed a second selection for affinity maturation using a different domain antibody (Vk (Y)) and a different cytokine (Cytokine X). The experimental execution of this experiment is highly similar to Example 7. As described in that example, an error-prone PCR library of >10⁸ variants based on Vk (Y), made using Genemorph II (Stratagene), was ligated in the pIE7t³T vector and PCR amplified with primers AS12 (SEQ ID no. 22) and AS18 (SEQ ID no. 24) to yield input material for the first round of selection. The error-rate of the library was determined by DNA sequencing of individual clones, obtained as described in Example 7, and was found to average 2.1 nucleotides per domain antibody gene.

Emulsion selections (i.e. emulsification, in vitro translation, breaking of emulsion, capture on streptavidin-coated PCR tubes, and PCR amplification of bound domain antibody DNA) were basically performed as described in Example 6, while the modifications mentioned in Example 7 were also applied in Example 8. The only differences were: 1) Cytokine X was used as cytokine, 2) no selections for improved off-rates were performed, and 3) no additional rounds of error-prone PCR were done during rounds of selection. A total of ten sequential rounds of selection were performed, during these rounds decreasing amounts of biotinylated Cytokine X were used: 50 nM in round 1; 35 nM in round 2; 20 nM in round 3; 15 nM in rounds 4 and 5; 10 nM in rounds 6, 7, and 8; 7.5 nM in round 9 and 5 nM in round 10.

After round 10, the selected domain antibodies were cloned SalI-NotI into a pUC119 based expression vector under control of the LacZ promoter (FIG. 12), and transformed to MACH1 cells (Invitrogen, Calif., USA). Ninety-six colonies were randomly picked and domain antibodies were expressed in supernatant. Screening of the supernatant in a Cytokine X ELISA identified domain antibodies with enhanced Cytokine X binding. These domain antibodies were purified for further characterisation and their affinity for Cytokine X was determined on a BIAcore1000.

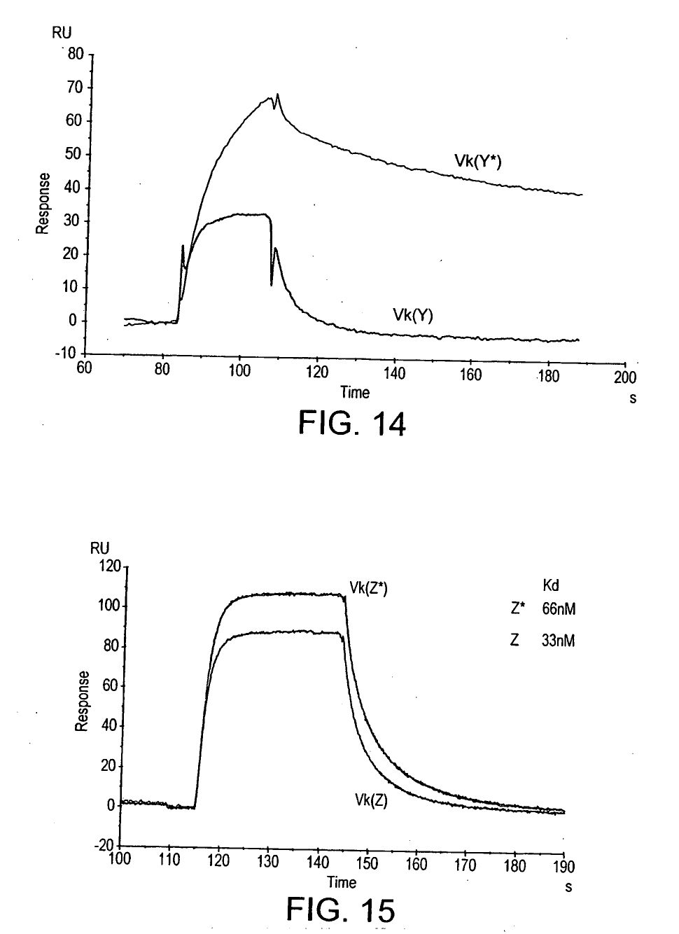

From this selection a domain antibody was identified (Vk (Y*)), with a single amino-acid mutation in CDR3, which resulted in a 25-fold improvement in affinity, as determined by BIAcore (FIG. 14). The BIAcore experiment was performed by injecting both parent and improved dAb, at the same concentration, over a Cytokine X coated BIAcore chip.

### Example 9

Affinity maturation of a Cytokine Y binding domain antibody using a TUS vector with a single TerB operator.

In the affinity maturation examples given so far, the vector used has always been pIE7t³T, which contains three TerB operators. Although three operators result in a tighter genotype-phenotype coupling, it might be beneficial to perform selections with a pure monovalent system which would contain only a single DNA operator. This would avoid any avidity components that might be associated with the use of three operators. Therefore, we also performed affinity maturation selections for a domain antibody against the Cytokine Y using a single TerB operator system.

Once again, as in Examples 7 and 8, a domain antibody (Vk (Z)) was amplified under error-prone PCR conditions and subsequently ligated in a TUS in vitro translation vector. This time though the vector used was pIE7tT, instead of pIE7t³T, having a single instead of three TerB operator sequences. The construction of this vector is described in Example 1 and the vector is shown in FIG. 3. Selections were performed as described in Examples 7 and 8, this time using eight rounds of selection and ligation in pIE7tT vector during each round of selection. Throughout these selection rounds, the breaking of the emulsions and the capture of the antigen on the streptavidin plates was always in the presence of at least 2 nM of free TerB operator. This is similar to Example 7, and is meant to scavenge any dissociating DNA-protein complexes. The Cytokine Y concentration was decreased during selection rounds as follows: 50 nM in round 1; 20 nM in round 2; 15 nM in round 3; 10 nM in rounds 4 and 5; 7.5 nM in rounds 6, 7, and 8. As described in Examples 7 and 8, the output of round 8 was cloned SalI-NotI in our expression vector, the dAbs expressed, and screened for improved binding. This identified a novel domain antibody (Vk (Z*)), containing a single mutation in CDR2, with a twofold improvement in affinity for Cytokine Y (FIG. 15). This improvement was determined by injection of both parent and improved variant, at the same concentration, on a BIAcore, where the chip surface had been coated with Cytokine.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### REFERENCES

Anderson, C. W., Straus, J. W. and Dudock, B. S. (1983) Methods Enzymol, 101, 635-44.
Anderson, J. E. (1993) Curr. Op. Struct. Biol., 3, 24-30.
Ash, M. and Ash, I. (1993) Handbook of industrial surfactants. Gower, Aldershot.
Barany, F. (1991) PCR Methods Applic., 1, 5-16.
Bass, S., Greene, R. and Wells, J. A. (1990) Proteins, 8, 309-14.
Becher, P. (1957) Emulsions: theory and practice. Reinhold, N.Y.
Benita, S., Ed. (1996). Microencapsulation: methods and industrial applications. Drugs and pharmaceutical sciences. Edited by Swarbrick, J. New York: Marcel Dekker.
Benner, S. A. (1994) Trends Biotechnol, 12, 158-63.
Blattner, F. R. and Dahlberg, J. E. (1972) Nature New Biol, 237, 227-32.
Bru, R. & Walde, P. (1991). Product inhibition of alpha-chymotrypsin in reverse micelles. Eur J Biochem 199(1), 95-103.
Bru, R. & Walde, P. (1993). Catalytic activity of elastase in reverse micelles. Biochem Mol Biol Int 31(4), 685-92.
Cahill, P., Foster, K. and Mahan, D. E. (1991) Clin Chem, 37, 1482-5.
Chakrabarti, A. C., Breaker, R. R., Joyce, G. F. & Deamer, D. W. (1994). Production of RNA by a polymerase protein encapsulated within phospholipid vesicles. J Mol Evol 39(6), 555-9.
Chang, T. M. (1987). Recycling of NAD(P) by multienzyme systems immobilized by microencapsulation in artificial cells. Methods Enzymol 136(67), 67-82.
Chang, T. M. S. (1992). Recent advances in artificial cells based on microencapsulation. In Microcapsules and nanoparticles in medicine and pharmacy (Donbrow, M., ed.), pp. 323-339. CRC Press, Boca Raton, Fla.
Chapman, K. B. and Szostak, J. W. (1994) Curr. op. Struct. Biol., 4, 618-622.
Choo Y, Klug A. (1993) 21(15):3341-6.
Chetverin, A. B. and Spirin, A. S. (1995) Prog Nucleic Acid Res Mol Biol, 51, 225-70.
Clackson, T. and Wells, J. A. (1994) Trends Biotechnol, 12, 173-84.
Creagh, A. L., Prausnitz, J. M. & Blanch, H. W. (1993). Structural and catalytic properties of enzymes in reverse micelles. Enzyme Microb Technol 15(5), 383-92.
Cull, M. G., Miller, J. F. and Schatz, P. J. (1992) Proc Natl Acad Sci USA, 89, 1865-9.
Dickinson, E. (1994) In Wedlock, D. J. (ed.), Emulsions and droplet size control. Butterworth-Heine-mann, Oxford, Vol. pp. 191-257.
Ellington, A. D. and Szostak, J. W. (1990) Nature, 346, 81822.
Ellman, J., Mendel, D., Anthony, C. S., Noren, C. J. and Schultz, P. G. (1991) Methods Enzymol, 202, 301-36.
Fahy, E., Kwoh, D. Y. and Gingeras, T. R. (1991) PCR Methods Appl, 1, 25-33.
Finch, C. A. (1993). Encapsulation and controlled release. Spec. Publ.-R. Soc. Chem. 138, 35.
Freese, E. (1959) J. Mol. Biol., 1, 87.
Friedberg, E. C., Walker, G. C. and Siede, W. (1995) DNA repair and mutagenesis. ASM Press, Washington D.C.
Gold, L., Polisky, B., Uhlenbeck, O. and Yarus, M. (1995) Annu Rev Biochem, 64, 763-97.
Green, R. and Szostak, J. W. (1992) Science, 258, 1910-5.
Gregoriadis, G. (1976) Methods Enzymol, 44, 21 8-27.
Griffiths, A. D., Williams, S. C., Hartley, O., Tomlinson, I. M., Waterhouse, P., Crosby, W. L., Kontermann, R. E., Jones, P. T., Low, N. M., Allison, T. J. and et al. (1994) Embo J. 13, 3245-60.
Haber, J., Maslakiewicz, P., Rodakiewicz, N. J. & Walde, P. (1993). Activity and spectroscopic properties of bovine liver catalase in sodium bis(2-ethylhexyl)sulfosuccinate/isooctane reverse micelles. Eur J Biochem 217(2), 567-73.
Hoogenboom, H. R. (1997) Trends Biotechnol., 15, 62-70.
Hopp T P, Pricket K S, Price V L, Libby R T, March C J, Ceretti D P, Urdal D L, Conlon P J (1988) Bio/Technology 6:1204-1210.
Janda, K. D., Lo, L.-C., Lo, C.-H. L., Sim, M.,-M., Wang, R., Wong, C.-H. and Lerner, R. A. (1997) Science, 275, 945-948.
Joyce, G. F. (1994) Curr. op. Structural Biol., 4, 331-336.
Karmirantzou M, Hamodrakas S J. (2001) Protein Eng. July; 14(7):465-72.
Katanaev, V. L., Kurnasov, O. V. and Spirin, A. S. (1995) Febs Lett, 359, 89-92.
Kowalczykowski, S. C., Dixon, D. A., Eggleston, A. K., Lauder, S. D. and Rehrauer, W. M. (1994) Microbiol Rev, 58, 401-65.
Kumar, A., Kumar, A. & Katiyar, S. S. (1989). Activity and kinetic characteristics of glutathione reductase in vitro in reverse micellar waterpool. Biochim Biophys Acta 996(1-2), 1-6.
Landegren, U., Kaiser, R., Sanders, J. and Hood, L. (1988) Science, 241, 1077-80.
Lesley, S. A. (1995) Methods Mol Biol, 37, 265-78.
Lesley, S. A., Brow, M. A. and Burgess, R. R. (1991) J Biol Chem, 266, 2632-8.
Leung, D. W., Chen, E. and Goeddel, D. V. (1989) Technique, 1, 11-15.
Liao, H., McKenzie, T. and Hageman, R. (1986) Proc Natl Acad Sci USA, 83, 576-80.
Lim, F. & Sun, A. M. (1980). Microencapsulated islets as bioartificial endocrine pancreas. Science 210(4472), 908-10.
Lim, F., Ed. (1984). Biomedical applications of microencapsulation. Boca Raton, Fla.: CRC Press.
Lissant, K. J., ed Emulsions and emulsion technology. Surfactant Science New York: Marcel Dekker, 1974.
Lissant, K. J., ed. Emulsions and emulsion technology. Surfactant Science New York: Marcel Dekker, 1974.
Lissant, K. J., ed. Emulsions and emulsion technology. Surfactant Science New York: Marcel Dekker, 1984.
Low, N. M., Holliger, P. H. and Winter, G. (1996) J Mol Biol, 260, 359-68.
Lowman, H. B., Bass, S. H., Simpson, N. and Wells, J. A. (1991) Biochemistry, 30, 10832-8.
Luisi, P. L. & B., S.-H. (1987). Activity and conformation of enzymes in reverse micellar solutions. Methods Enzymol 136(188), 188-216.
Manley, J. L., Fire, A., Samuels, M. and Sharp, P. A. (1983) Methods Enzymol, 101, 568-82.
Mao, Q. & Walde, P. (1991). Substrate effects on the enzymatic activity of alpha-chymotrypsin in reverse micelles. Biochem Biophys Res Commun 178(3), 1105-12.
Mao, Q., Walde, P. & Luisi, P. L. (1992). Kinetic behaviour of alpha-chymotrypsin in reverse micelles. A stopped-flow study. Eur J Biochem 208(1), 165-70.
Mattheakis, L. C., Bhatt, R. R. and Dower, W. J. (1994) Proc Natl Acad Sci USA, 91, 9022-6.
McCafferty, J., Griffiths, A. D., Winter, G. and Chiswell, D. J. (1990) Nature, 348, 552-4.
Melton, D. A., Krieg, P. A., Rebagliati, M. R., Maniatis, T., Zinn, K. and Green, M. R. (1984) Nucleic Acids Res, 12, 703556.
Mendel, D., Cornish, V. W. and Schultz, P. G. (1995) Annu Rev Biophys Biomol Struct, 24, 435-62.
Menger, F. M. & Yamada, K. (1979). J. Am. Chem. Soc. 101, 6731-6734.
Miele, E. A., Mills, D. R. and Kramer, F. R. (1983) J Mol Biol, 171, 281-95.
Miller J, McLachlan A D, Klug A. (1984) EMBO J. 1985 June; 4(6):1609-14.
Moore, M. J. (1995) Nature, 374, 766-7.
Moore M, Choo Y, Klug A. (2001) 98(4), 1432-6.
New, R. R. C., Ed. (1990). Liposomes: a practical approach. The practical approach series. Edited by Rickwood, D. & Hames, B. D. Oxford: Oxford University Press.
Nissim, A., Hoogenboom, H. R. I Tomlinson, I. M., Flynn, G., Midgley, C., Lane, D. and Winter, G. (1994) Embo J, 13, 692-8.
Oberholzer, T., Albrizio, M. & Luisi, P. L. (1995a). Polymerase chain reaction in liposomes. Chemistry and Biology 2, 677-682.
Oberholzer, T., Wick, R., Luisi, P. L. & Biebricher, C. K. (1995b). Enzymatic RNA replication in self-reproducing vesicles: an approach to a minimal cell. Biochem Biophys Res Commun 207(1), 250-7.
Parmley, S. F. and Smith, G. P. (1988) Gene, 73, 305-18.
Pelham, H. R. and Jackson, R. J. (1976) Eur J Biochem, 67, 247-56.
Perelson, A. S. and Oster, G. F. (1979) J Theor Biol, 81, 64570.
Perez, G. M., Sanchez, F. A. & Garcia, C. F. (1992). Application of active-phase plot to the kinetic analysis of lipoxygenase in reverse micelles. Biochem J.
Raumann, B. E., Rould, M. A., Pabo, C. O. And Sauer, R. T. (1994) Nature 367, 6465, 754-7.
Roberts, B. E., Gorecki, M., Mulligan, R. C., Danna, K. J., Rozenblatt, S, and Rich, A. (1975) Proc Natl Acad Sci USA, 72, 1922-6.
Roberts, J. W. (1969) Nature, 224, 1168-74.
Roberts, R. & Szostak, J. (1997) RNA-peptide fusions for the in vitro selection of peptides and proteins. Proc Natl Acad Sci USA 94, 12297-12302.
Rosenberg, M., Weissman, S. and Decrombrugghe, B. (1975) J Biol Chem, 250, 4755-64.
Ryabova, L. A., Desplancq, D., Spirin, A. S. And Pluckthun (1997) Nat Biotechnol 15(1): 79-84.
Saiki, R. K., Gelfand, D. H., Stoffel, S., Scharf, S. J., Higuchi, R., Horn, G. T., Mullis, K. B. and Erlich, H. A. (1988) Science, 239, 487-91.
Sambrook, J., Fritsch, E. F. and Maniatis, T. (1989) Molecular cloning: a laboratory manual. Cold Spring Harbor Laboratory Press, New York.
Sherman, P. (1968) Emulsion science. Academic Press, London.
Smith, G. P. (1985) Science, 228, 1315-7.
Soumillion, P., Jaspers, L., Bouchet, M., Marchand, B. J., Winter, G. and Fastrez, J. (1994) J Mol Biol, 237, 415-22.
(Speight, R. E., Hart, D. J., Sutherland, J. D. and Blackburn, J. M. (2001) Chem & Biol 8, 951-956.
Stemmer, W. P. (1994a) Nature, 370, 389-91.
Stemmer, W. P. (1994b) Proc Natl Acad Sci USA, 91, 10747-51.
Stofko, H. R., Carr, D. W. and Scott, J. D. (1992) Febs Lett, 302, 274-8.
Sun, A. M., Vasek, I. & Tai, I. (1992). Microencapsulation of living cells and tissues. In Microencapsulation and nanoparticles in medicine and pharmacy (Donbrow, M., ed.), pp. 315-322. CRC Press, Boca Raton, Fla.
Tuerk, C. and Gold, L. (1990) Science, 249, 505-10.
van Hal, D. A., Bouwstra, J. A. & Junginger, H. E. (1996). Nonionic surfactant vesicles containing estradiol for topical application. In Microencapsulation: methods and industrial applications (Benita, S., ed.), pp. 329-347. Marcel Dekker, New York.
Walde, P., Goto, A., Monnard, P.-A., Wessicken, M. & Luisi, P. L. (1994). Oparin's reactions revisited: enzymatic synthesis of poly(adenylic acid) in micelles and self-reproducing vesicles. J. Am. Chem. Soc. 116, 7541-7547.
Walde, P., Han, D. & Luisi, P. L. (1993). Spectroscopic and kinetic studies of lipases solubilized in reverse micelles. Biochemistry 32(15), 4029-34.
Walde, P., Peng, Q., Fadnavis, N. W., Battistel, E. & Luisi, P. L. (1988). Structure and activity of trypsin in reverse micelles. Eur J Biochem 173(2), 401-9.
Walker, G. T., Fraiser, M. S., Schram, J. L., Little, M. C., Nadeau, J. G. and Malinowski, D. P. (1992) Nucleic Acids Res, 20, 1691-6.
Weil, P. A., Luse, D. S., Segall, J. and Roeder, R. G. (1979) Cell, 18, 469-84.
Whateley, T. L. (1996). Microcapsules: preparation by interfacial polymerisation and interfacial complexation and their applications. In Microencapsulation: methods and industrial applications (Benita, S., ed.), pp. 349-375. Marcel Dekker, New York.
Wick, R. & Luisi, P. L. (1996). Enzyme-containing liposomes can endogenously produce membrane-constituting lipids. Chem Biol 3(4), 277-85.
Widersten, M. and Mannervik, B. (1995) J Mol Biol, 250, 115-22.
Winter, G., Griffiths, A. D., Hawkins, R. E. and Hoogenboom, H. R. (1994) Annu Rev Immunol, 12, 433-55.
Yamagishi, J., Kawashima, H., Matsuo, N., Ohue, M., Yamayoshi, M., Fukui, T., Kotani, H., Furuta, R., Nakano, K. and Yamada, M. (1990) Protein Eng, 3, 713-9.
Yelamos, J., Klix, N., Goyenechea, B., Lozano, F., Chui, Y. L., Gonzalez, F. A., Pannell, R., Neuberger, M. S, and Milstein, C. (1995) Nature, 376, 225-9.
Zubay, G. (1973) Annu Rev Genet, 7, 267-87.
Zubay, G. (1980) Methods Enzymol, 65, 856-77.

1. A nucleotide sequence encoding a Tus DNA binding domains, a DNA binding sites and a polypeptide domain wherein the nucleotide sequence is compartmentalised in a capsule.

2. A nucleotide sequence encoding a Tus DNA binding domains, a DNA binding sites and a polypeptide domain wherein the polypeptide domain is an antibody domain.

3. A nucleotide sequence according to claim 2, wherein the antibody domain is a VL, V_{H}, V_{H} or Camelid V_{HH} domain.

4. A nucleotide sequence according to claims 1 or 2, wherein the nucleotide sequence comprises a tag sequence.

5. A nucleotide sequence according to claim 4, wherein the tag sequence is included at the 3′ end of the nucleotide sequence.

6. A nucleotide sequence according to claim 4 or claim 5, wherein the tag sequence is selected from the group consisting of HA, FLAG or c-Myc.

7. A nucleotide sequence according to claim 5, wherein the tag sequence is selected from the group consisting of HA, FLAG or c-Myc.

8. A nucleotide sequence according of claims 1 or 2, wherein the polypeptide domain is fused directly or indirectly to the N-terminus of the Tus DNA binding domain(s).

9. A nucleotide sequence according of claims 1 or 2, wherein the Tus DNA binding domain(s) comprises or consists of the sequence set forth in Seq ID No. 1 or Seq ID No. 2.

10. A nucleotide sequence according to any of the preceding claims, wherein the nucleotide sequence additionally comprises a linkers.

11. A nucleotide sequence according to any one of the preceding claims, wherein said nucleotide sequence comprises 1, 2 or 3 DNA-binding sites.

12. A nucleotide sequence according to any one of the preceding claims, wherein the a DNA-binding sites are Ter operator(s).

13. A nucleotide sequence according to claim 11, wherein the Ter operator(s) comprise or consist of TerB.

14. A nucleotide sequence according to claim 11 or claim 12, wherein TerB comprises or consists of the sequence set forth in Seq ID No 3 or Seq ID No 4.

15. A nucleotide sequence according to any one of claims 3-13, wherein the antibody V_{L} domain is V_{K}.

16. A construct comprising the nucleotide sequence according to any one of claims 1-14

17. A vector comprising the nucleotide sequence according to any one of claims 1-14.

18. A host cell comprising the construct according to claim 15 or the vector according to claim 15.

19. A protein encoded by the nucleotide sequence according to any one of claims 1-14.

20. A protein-DNA complex comprising the protein according to claim 18 bound to a nucleotide sequence according to any of claims 1-14.

21. A method for preparing a protein-DNA complex according to claim 19, comprising the steps of:
(a) providing a nucleotide sequence according to any one claims 1 to 14, a construct according to claim 15 or a vector according to claim 16; and
(b) expressing the nucleotide sequence to produce its respective protein; and
(c) allowing for the formation of the protein-DNA complex.

22. A method for isolating a nucleotide sequences encoding a polypeptide domain with a desired specificity, comprising the steps of:
(a) providing a nucleotide sequence according to any one claims 1 to 14, a construct according to claim 15 or a vector according to claim 16;
(b) compartmentalising the nucleotide sequence into microcapsules;
(c) expressing the nucleotide sequence to produce its respective polypeptide domain;
(d) pooling the microcapsules into a common compartment; and
(e) selecting the nucleotide sequence which produces a polypeptide domain having the desired specificity.

23. A method according to any one of claim 21 further comprising the additional step of:
(f) introducing a mutations into the polypeptide domain.

24. A method according to claim 21 or claim 22 further comprising iteratively repeating a of steps (a) to (e).

25. A method according to any one of claims 21-23 further comprising amplifying the polypeptide domain.

26. A method according to any one of claims 21-24, wherein the polypeptide domain(s) are sorted by affinity purification.

27. A method according to claim 25 wherein the polypeptide domain(s) are sorted using protein L.

28. A method according to any one of claims 21 to 26, wherein the polypeptide domains are sorted by selective ablation of polypeptide domains, which do—riot encode the desired polypeptide domain gene product.

29. A method for preparing a polypeptide domain, comprising the steps of:
(a) providing a nucleotide sequence according to any one claims 1 to 14, a construct according to claim 15 or a vector according to claim 16;
(b) compartmentalising the nucleotide sequences;
(c) expressing the nucleotide sequences to produce their respective gene products;
(d) sorting the nucleotide sequences which produce polypeptide domains having the desired specificity; and
(e) expressing the polypeptide domains having the desired specificity.

30. A protein-DNA complex obtained or obtainable by the method according to claim 20.

31. Use of a Tus DNA binding domains and/or a Ter DNA binding sites in the selection of a polypeptide domain.


SEQUENCE LISTING (35 sequences)

SEQ ID NO: 1 - protein, 309 aa, Escherichia coli [sequence omitted]

SEQ ID NO: 2 - DNA, 2079 bp, Escherichia coli
tggcataacatcccgcaatttacctctgcctgacactacgcgcacgatggtcaagtcaccacgactgtgctataaaataagtatgttgtaactaaagtggttaatattatggcgcgttacgatctcgtagaccgactcaacactacctttcgccagatggaacaagagctggctatatttgccgctcatcttgagcaacacaagctattggttgcccgcgtgttctctttgccggaggtaaaaaaagaggatgagcataatccgcttaatcgtattgaggtaaaacaacatctcggcaacgacgcgcagtcgctggcgttgcgtcatttccgccatttatttattcaacaacagtccgaaaatcgcagcagcaaggccgctgtccgtctgcctggcgtgttgtgttaccaggtcgataacctttcgcaagcagcgttggtcagtcatattcagcacatcaataaactcaagaccacgttcgagcatatcgtcacggttgaatcagaactccccaccgcggcacgttttgaatgggtgcatcgtcatttgccggggctgatcacccttaatgcttaccgcacgctcaccgttctgcacgaccccgccactttacgctttggttgggctaataaacatatcattaagaatttacatcgtgatgaagtcctggcacagctggaaaaaagcctgaaatcaccacgcagtgtcgcaccgtggacgcgcgaggagtggcaaagaaaactggagcgagagtatcaggatatcgctgccctgccacagaacgcgaagttaaaaatcaaacgtccggtgaaggtgcagccgattgcccgcgtctggtacaaaggagatcaaaaacaagtccaacacgcctgccctacaccactgattgcactgattaatcgggataatggcgcgggcgtgccggacgttggtgagttgttaaattacgatgccgacaatgtgcagcaccgttataaacctcaggcgcagccgcttcgtttgatcattccacggctgcacctgtatgttgcagattaacgcccggctttcatactgccgaccatctgttctggccgtacccagctgtcaaactcggcttcgctaagataccccagcgcaagggccgcagcttttaaggtcagcccttctttatgcgcttttttggcgatctcggcggctttgtcataaccaatgtgggtgttaagcgcagtcaccagcatcagcgattcattgagtaattgattgattcgctcacgattcggttcaatacccactgcgcagtgtttgttaaaactttccatgccatctgccagcaagcgcaccgattgcaggaaattgtggatcaccattggacggaagacgttcagttcaaagttaccggaagcgccccccatgttgatcgccacgtcgttccccatcacctgacagcagagcatggttaatgcctcacactgtgttgggttcactttccccggcatgattgagctgcccggctcattttccgggattgagatttcaccaattcgcagcgcggccagaggccagccagcggacatcattggcgattttcatcagtgacgcagccaaccctttcaaccgccgtgcgcctgaaccagggcatcacaggtcgccagcgcttcaaatttgttcggcgcggtaacaaacggtgcacaggtaatgactgccagttcatctgctacgcgacgcgcatactccggatgggtatttagtccagtacccaccgctgtaccgccaagagccagttccgctacgtgaggcaggctgtattcgatatgtttgagattatgctcgagcatcgctacccagccggaaatctcctgccccagcgttaacggcgtggcatcctgcaagtgagtacgaccaattttgacgatatcgcaaaagcacgggatttctcattcagtgtctgtgtcagggttttaagctgaggaatgagttgcttgcgcagcgccagcagcgccgcaacgagcatcgccgtcggaaagacatcgttggaactttggcttttgttcacgtcgtcgttaggtgaactttacgttccatcccgcgcacaccgcc

SEQ ID NO: 3 - DNA, 23 bp, Escherichia coli
aataagtatgttgtaactaaagt

SEQ ID NO: 4 - DNA, 16 bp, Artificial sequence
  Consensus nucleotide sequence of TerB
  misc_feature (1, 3, 5, 16): n = a, t, g or c
ngnrngttgtaaykan

SEQ ID NO: 5 - DNA, 346 bp, Artificial sequence
  Synthetic sequence Vkappa(TAR1-5-19)
ccatggcgtcgacggacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgggcggccgc

SEQ ID NO: 6 - DNA, 324 bp, Artificial sequence
  Synthetic sequence Vkappa(TAR1-5)
gacatccagatgacccagtctccatcctccctgtctgcatctgtaggagaccgtgtcaccatcacttgccgggcaagtcagagcatttttatgaatttattgtggtaccagcagaaaccagggaaagcccctaagctcctgatctataatgcatccgtgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 7 - DNA, 346 bp, Artificial sequence
  Synthetic sequence Vkappa(E5)
ccatggcgtcgacggacatccagatgacccagtctccatcctccctgtctgcatctgtaggagacagagtcaccatcacttgccgggcaagtcagagcgttagcagctatttaaattggtatcagcagaaaccagggaaagcccctaagctcctgatctatcttgcatcccgtttgcaaagtggggtcccatcaaggttcagtggcagtggatctgggacagatttcactctcaccatcagcagtctgcaacctgaagattttgcaacttactactgtcaacagaattggtggctgcctcctacgttcggccaagggaccaaggtggaaatcaaacgggcggccgc

SEQ ID NO: 8 - DNA, 130 bp, Artificial sequence
  Synthetic sequence KEA linker
gcggccgcaaaagaagcggcggcgaaagaagcggcggcgaaagaagcggcggcgaaagaattggccgcaaaagaagcggcggcgaaagaagcggcggcgaaagaagcggcggcgaaagaattggccgcag

SEQ ID NO: 9 - protein, 43 aa, Artificial sequence
  Synthetic sequence KEA linker
AAAKEAAAKEAAAKEAAAKELAAKEAAAKEAAAKEAAAKELAA

SEQ ID NO: 10 - DNA, 21 bp, Artificial sequence
  Synthetic sequence Oligonucleotide AS5
catggggtcgaccggcggcgc

SEQ ID NO: 11 - DNA, 21 bp, Artificial sequence
  Synthetic sequence Oligonucleotide AS6
ggccgcgccgccggtcgaccc

SEQ ID NO: 12 - DNA, 56 bp, Artificial sequence
  Synthetic sequence Oligonucleotide AS1
catggggtcgaccatgcatggcggccgcaggatcccatcatcatcaccatcactaa

SEQ ID NO: 13 - DNA, 56 bp, Artificial sequence
  Synthetic sequence Oligonucleotide AS2
gatcttagtgatggtgatgatgatgggatcctgcggccgccatgcatggtcgaccc

SEQ ID NO: 14 - DNA, 28 bp, Artificial sequence
  Synthetic sequence Primer AS102
cgcggatccatggcgcgttacgatctcg

SEQ ID NO: 15 - DNA, 33 bp, Artificial sequence
  Synthetic sequence Primer AS103
gagagaagatctatctgcaacatacaggtgcag

SEQ ID NO: 16 - DNA, 42 bp, Artificial sequence
  Synthetic sequence Oligonucleotide AS105
gatctcactttagttacaacatacttattttctctctctctg

SEQ ID NO: 17 - DNA, 42 bp, Artificial sequence
  Synthetic sequence Oligonucleotide AS114
gatccagagagagagaaaataagtatgttgtaactaaagtga

SEQ ID NO: 18 - DNA, 19 bp, Artificial sequence
  Synthetic sequence Primer AS16
ctgccaccatacccacgcc

SEQ ID NO: 19 - DNA, 60 bp, Artificial sequence
  Synthetic sequence AS120
ggccaaagaagcggcggcgaaagaagcggcggcgaaagaagcggcggcgaaagaagccgc

SEQ ID NO: 20 - DNA, 60 bp, Artificial sequence
  Synthetic sequence AS121
ggccgcggcttctttcgccgccgcttctttcgccgccgcttctttcgccgccgcttcttt

SEQ ID NO: 21 - DNA, 20 bp, Artificial sequence
  Synthetic sequence AS11
ttcgctattacgccagctgg

SEQ ID NO: 22 - DNA, 20 bp, Artificial sequence
  Synthetic sequence Primer AS12
aaagggggatgtgctgcaag

SEQ ID NO: 23 - DNA, 19 bp, Artificial sequence
  Synthetic sequence Primer AS17
cagtcaggcaccgtgtatg

SEQ ID NO: 24 - DNA, 21 bp, Artificial sequence
  Synthetic sequence Primer AS18
aacaatgcgctcatcgtcatc

SEQ ID NO: 25 - DNA, 26 bp, Artificial sequence
  Synthetic sequence Primer OA16
ataccatggggtcgacggacatccag

SEQ ID NO: 26 - DNA, 31 bp, Artificial sequence
  Synthetic sequence Primer OA17N
ttcttttgcggccgcccgtttgatttccacc

SEQ ID NO: 27 - DNA, 20 bp, Artificial sequence
  Synthetic sequence AS92
ttcgctattacgccagctgg

SEQ ID NO: 28 - DNA, 23 bp, Artificial sequence
  Synthetic sequence Oligonucleotide AS87N
tgtggtctccctatagtgagtcg

SEQ ID NO: 29 - DNA, 24 bp, Artificial sequence
  Synthetic sequence DOM8
agcggataacaatttcacacagga

SEQ ID NO: 30 - DNA, 24 bp, Artificial sequence
  Synthetic sequence DOM9
cgccagggttttcccagtcacgac

SEQ ID NO: 31 - DNA, 287 bp, Artificial sequence
  Synthetic sequence Fragment of pIE2
  CDS (103)..(165)
tcgagatctcgatcccgcgaaattaatacgactcactatagggagaccacaacggtttccctctagaaataattttgtttaactttaagaaggagatataccatggggtcgaccggcggcgcggccgcaggatcctatccgtatgatgtgccggattatgcgtaactcgagagatccggtaacaaagcccgaaaggaagctgagttggctgctgccaccgctgagcaataactagcataaccccttggggcctctaaacgggtcttgaggggttttttgctgaaagg

SEQ ID NO: 32 - protein, 20 aa, Artificial sequence
  Synthetic sequence Fragment of pIE2
MGSTGGAAAGSYPYDVPDYA

SEQ ID NO: 33 - DNA, 287 bp, Artificial sequence
  Synthetic sequence Fragment of pIE2
cctttcagcaaaaaacccctcaagacccgtttagaggccccaaggggttatgctagttattgctcagcggtggcagcagccaactcagcttcctttcgggctttgttaccggatctctcgagttacgcataatccggcacatcatacggataggatcctgcggccgcgccgccggtcgaccccatggtatatctccttcttaaagttaaacaaaattatttctagagggaaaccgttgtggtctccctatagtgagtcgtattaatttcgcgggatcgagatctcga

SEQ ID NO: 34 - protein, 47 aa, Artificial sequence
  Consensus sequence for the TFIIIA-type zinc finger domains
  MISC_FEATURE (1)..(1): Xaa is Tyr or Phe
  MISC_FEATURE (2, 4..27, 29..31, 33..37, 39, 40, 42..46): Xaa is any amino acid
  MISC_FEATURE (42)..(46): Any 2 Xaas may be present or absent - indicates a range of 3-5 amino acids
XXCXXXXXXXXXXXXXXXXXXXXXXXXCXXXFXXXXXLXXHXXXXXH

SEQ ID NO: 35 - protein, 9 aa, Human influenza virus
YPYDVPDYA